SEQUENCE LISTING (12 sequences)

SEQ ID NO: 1 - DNA, 969 bp, Gluconobacter oxydans
atgtcccagcgcatcaccatcgatcaccgttcggcgcctgcccttcgccatccggagaaggcgcatcggcctgataacccgatccagcgcaagccgtcctggatccgggtcaaggcgccgaaccatccagtctatcacgagacccgtgcgctcatgcgggatgccgggctggtgacggtctgcgaggaggcggcgtgccccaatatcggggaatgctggtcgcagcgtcatgccacgatgatgatcatgggcgagatctgcacccgggcgtgtgcattctgcaatgtcacgaccggactgccgaagcatctcgatgaggacgagccacggcgggtcggagaagcggtggcgaagcttgggctcaagcatgtcgtcatcacttcggtggaccgtgacgatctggaagacgggggggcgatgcacttcgcgcgggtcattcatgcgatccgcgagacctctccacagaccacgatcgagattctgacgcctgatttcctgcgcaaggatggcgcgctggaggtcgtggtggcggctcggccggacgttttcaatcacaatatcgagaccataccccggctttaccccaccatcagaccgggtgcgcgttactatcagtccgtccgtctcctggacggagtaaagaaactcgatccttccatttttaccaagtccggcctgatgcttgggcttggcgaggagcggatggaagtggcccaggtgatggatgatttccggatcgcggacgtcgatttcctgacgcttggacagtatctccagccgtccgcgaaacatgcggcggtggaaaagtttgttacccccgacgagtttgacggctatgctgctgctgcccgttcaaaaggatttcttcaggtcagtgccagtccgctgacccggtcctcctaccatgcggacagtgatttcgcgaagcttcaggctgcgcggaacagccgtctgaaagaaagtctctga

SEQ ID NO: 2 - DNA, 702 bp, Gluconobacter oxydans
atgccgaaaacgagggcaatgacccgaaatgagatttgtgaagaaattttgtggaaatcttccccgggactgactccctaccccgaggccctgaccttcatggaggagcgtgcgcgggccattcatcaggggacagcagaaccgctggtctggcttgtcgagcatcctcccgtctttacggcaggcacctccgccaaagatgcggacctctacaatcctcacggctacccgacctattccgccggacgcggcggccagtggacctatcacggaccggggcaaaggctcggttatgtcatgatggatctgacgaagccgaacggcaccgtcccgtcccgcgacctgcgcgcttttgtcgcgggactggaaggctggatgaccgacaccctcgcccggctcggcgtcacggcctttacccgagagggccggatcggcgtctggacgatcgatcccctcacgggcctggaagccaagatcggggcgctgggcatccgcgtcagccggtgggtcagctggcatggcgtttcgatcaatgtcagtcccgatctgacagatttcgatggaatcgtgccctgcggcatccgcgagttcggtgtcaccagtctccagcgattcgacagcagcctgacgatggcggatctcgatgacgccctcgccgccgcatggcccggacggttcggctctattccgcaggcggcgtga

SEQ ID NO: 3 - DNA, 435 bp, Gluconobacter oxydans
gtgagtgatgcctatcttgttccccaggaggacacggctgctgcggctatcgaggagatcgaggccgagctgggtctgtttgatgactggatggagcggtatcagtacatcatcgagatgggacgcaagctgccgccatttccggaagagtggcaggatgatgcccatcgggttccgggctgtcagagccaggtctggcttgaagcggtcgaacgggatggaaagctgtttttcgccggggcgtcggatgccgcgatcgttcaggggctcgtggcgcttctgctgagggtttattccggccgtccgaaatcggaaattctgggaacaagccctgtgtttctgcatgacatgggactggtcaaggcgctttcgacgaaccgtggcaacggggtcgaggctatggcgcaggccattcagaagcgcgcgtcacactag

SEQ ID NO: 4 - DNA, 969 bp, Artificial Sequence
  source: /note=“Description of Artificial Sequence Synthetic polynucleotide”
atgtcccagcgcatcacgatcgaccatcgctccgccccggccctgcgccatccggaaaaggcccatcgcccggacaacccgatccagcgcaagccgtcctggatccgcgtcaaggccccgaaccatccggtctatcatgaaacgcgcgccgtcatgcgcgacgccggcgtcgtcacggtctgcgaagaagccgcctgcccgaacatcggcgaatgctggtgccaggtccatgccacgatgatgatcatgggcgaaatctgcacgcgcgcctgcgccttctgcaacgtcacgacgggcgtcccgaagcatgtcgacgaagacgaaccgcgccgcgtcggcgaagccgtcgccaagctgggcgtcaagcatgtcgtcatcacgtccgtcgaccgcgacgacgtcgaagacggcggcgccatgcatttcgccggcgtcatccatgccatccgcgaaacgtccccgcagacgacgatcgaaatcgtcacgccggacttcgtccgcaaggacggcgccgtcgaagtcgtcgtcgccgccggcccggacgtcttcaaccataacatcgaaacgatcccgggcctgtatccgacgatccgcccgggcgcccgctattatcagtccgtccgcgtcgtcgacggcgtcaagaaggtcgacccgtccatcttcacgaagtccggcgtcatgctgggcctgggcgaagaacgcatggaagtcgcccaggtcatggacgacttcggcatcaccgacgtcgacttcgtcacgctgggccagtatctccagccgtccgccaagcatgccgccgtcgaaaagttcgtcacgccggacgaattcgacggctatgccgctgctgcccgttcaaaaggatttcttcaggtcagtgccagtccgctgacccggtcctcctaccatgcggacagtgatttcgcgaagcttcaggctgcgcggaacagccgtctgaaagaaagtctctga

SEQ ID NO: 5 - DNA, 702 bp, Artificial Sequence
  source: /note=“Description of Artificial Sequence Synthetic polynucleotide”
atgccgaagacgcgcgccatgacgcgcaacgaaatctgcgaagaaatcctgtggaagtcctccccgggcgtcacgccgtatccggaagccgtcacgttcatggaagaacgcgccggcggcatccatcagggcacggccgaaccgggcgtctggctggtcgaacatccgccggtcttcacggccggcacgtccgccaaggacgccgacgtctataacccgcatggctatccgacgtattccgccggccgcggcggccagtggacgtatcatggcccgggccagcgcgtcggctatgtcatgatggacggcacgaagccgaacggcacggtcccgtcccgcgacctgcgcgccttcgtcgccggcgtcgaaggctggatgacggacacggtcgccggcgtcggcgtcacggccttcacgcgcgaaggcggcatcggcgtctggacgatcgacccggtcacgggcggcgaagccaagatcggcgccggcggcatccgcgtcgccggctgggtcgcctggcatggcgtctccatcaacgtctccccggacggcacggacttcgacggcatcgtcccgtgcggcatccgcgaattcggcgtcacgtccgtccagcgcttcgacgccgccggcacgatggccgacgtcgacgacgccgtcgccgccgcctggccgggcggcttcggctccatcccgcaggccgcctga

SEQ ID NO: 6 - DNA, 435 bp, Artificial Sequence
  source: /note=“Description of Artificial Sequence Synthetic polynucleotide”
gtctccgacgcctatctggtcccgcaggaagacacggccgccgccgccatcgaagaaatcgaagccgaactgggcctgttcgacgactggatggaacgctatcagtatatcatcgaaatgggccgcaagctgccgccgttcccggaagaatggcaggacgacgcccatcgcgtcccgggctgccagtcccaggtctggctggaagccgtcgaacgcgacggcaagctgttcttcgccggcgcctccgacgccgccatcgtccagggcctggtcgccctgctgctgcgcgtctattccggccgcccgaagtccgaaatcctgggcacgtccccggtcttcctgcatgacatgggcctggtcaaggccctgtccacgaaccgcggcaacggcgtcgaagccatggcccaggccatccagaagcgcgcctcccattag

SEQ ID NO: 7 - DNA, 924 bp, Gluconobacter oxydans
atgacgaccggaaacatcccagacccgtcatcgcctgcgaaaggcccggtctgcatcatcggcgccagcggccgctcaggatccgcgctctgccgcgcgcttctggctgaaggccagaagatcattgccgtcgtgcgcagccagggcaatctcgcgcccgacatcgcagaagcctgtcaagccgtgcggatcgcagaccttacggacagcgccacgctggctctggcgttcgaagatgcggcggttatcgtcaacacggcccatgcgcgacacttgcccgccattctggccgctacgaaagcccctatcgtagcgctgggcagcacacgcaaattcacgcgctggcccgacgatcacggacggggtgtcctcgcaggcgaaaccgccctcaaggccgacggtcgcccgtcgatcatcctgcatccgaccatgatctatggtgcccagggcgaagacaatgtgcagcggctagcgaagctgctggagcgcctgcccgtcatcccacttcctggcggtggccgtgccctcgtgcagcccatcgaccagcgggacgttacacgctgcctggtctcggccatacatctgatccagaacggagacgtcacggggccggaaagcatcgtgatcgccggtccgacagcggtggcttaccggaccttcgtgcgcatggtgctgtattttgcgggacttggcggccgtcccatcgtctccctgccgggatggatgctcatggcgctgtctcatctgacgcggcatatccgcagactgccgcagatcgcgccggaagaaatccgccgccttctggaagacaagaatttcgatgtgggtccgatggaacagcgtctgggcgtcacgcccgttccgcttgccaacgggctgcaccatctgttcggcaacagaacgcgccagaagcaggagccctga

SEQ ID NO: 8 - DNA, 477 bp, Gluconobacter oxydans
atgaccacgaaatttgaactcaacggacagcccgttacggtcgacgccccggcagacacccccctgctgtgggtcatccgtgacgacctgaacctgaccggcaccaagttcggctgcggcatcggcgaatgcggtgcctgcaccgtccatgtgggcggccgcgccacgcgctcctgcatcacgccgctctccgccgtcgaaggcgcttcgatcaccacgatcgaaggcctcgacccggcaggaaaccacgtcgtgcaggtcgcctggcgtgaccagcaggtgccgcagtgcggctactgccagtccggccagatcatgcaggccgcaagcctcctgaaggactatccgaacccgacggacgaccagattgacggcgtaatgggcggcagcctctgccgctgcatgacctatatccgcatccgcaaggccatcaaggaagctgcctcaaggcagcaggaaggcgccaacaatggctaa

SEQ ID NO: 9 - DNA, 819 bp, Gluconobacter oxydans
tcagaagcggtattcgatacctgcgccaacgacggtcgggtcgagggaatcatgcgccgtgatcttcgtcgtctcggcactgtcctgggcccagacgtggacacgcatgaacatctgcttcacgtcgaagttgaagaaccagttgccgacgacctggtagtcgaagcctacattgacggacgggccaccggtcacaccgatgttcaccttctgcgtcaggccattgttggccggagagatgtcgtggaaccatgccagcgttgcgccaacacccacatacggattgaagcgcttgtgcgggcggaagtgccaggcgaacgtgaccgtcggcggcagaacccaggcgctgccaacatcgaccttgcccagacccggaacgccttcagcggcgatctcgtgacgggtgctggcggcgatcaggtcaaccgacaggttatccgtgaagaaatattcgaacgtcagttccggcatgacctgacgcgtcgtcttcacgcggccaccgagatgggcgccgttcagggacaggctgctgtcacggtcttccggcagaacgccgagagcggacagacggacgatgaagtcgcccttgccgagtccgatgcgcgtgttggcgcaggtctggaacaggccgcagcgacccgaagccggcgcatggatgttcacggaaggcaccggagccgccgtcatgggagcactgaccatgacctgaggagcagcaggagcagttgccggggcggtctgggcagggaccgtctgggcaaacgccggagttgcaacaacaccgactgcggcagccagcgcaaaagcgttaaatttcat

SEQ ID NO: 10 - protein, 322 aa, Gluconobacter oxydans
MSQRITIDHRSAPALRHPEKAHRPDNPIQRKPSWIRVKAPNHPVYHETRALMRDAGLVTVCEEAACPNIGECWSQRHATMMIMGEICTRACAFCNVTTGLPKHLDEDEPRRVGEAVAKLGLKHVVITSVDRDDLEDGGAMHFARVIHAIRETSPQTTIEILTPDFLRKDGALEVVVAARPDVFNHNIETIPRLYPTIRPGARYYQSVRLLDGVKKLDPSIFTKSGLMLGLGEERMEVAQVMDDFRIADVDFLTLGQYLQPSAKHAAVEKFVTPDEFDGYAAAARSKGFLQVSASPLTRSSYHADSDFAKLQAARNSRLKESL

SEQ ID NO: 11 - protein, 233 aa, Gluconobacter oxydans
MPKTRAMTRNEICEEILWKSSPGLTPYPEALTFMEERARAIHQGTAEPLVWLVEHPPVFTAGTSAKDADLYNPHGYPTYSAGRGGQWTYHGPGQRLGYVMMDLTKPNGTVPSRDLRAFVAGLEGWMTDTLARLGVTAFTREGRIGVWTIDPLTGLEAKIGALGIRVSRWVSWHGVSINVSPDLTDFDGIVPCGIREFGVTSLQRFDSSLTMADLDDALAAAWPGRFGSIPQAA

SEQ ID NO: 12 - protein, 144 aa, Gluconobacter oxydans
VSDAYLVPQEDTAAAAIEEIEAELGLFDDWMERYQYIIEMGRKLPPFPEEWQDDAHRVPGCQSQVWLEAVERDGKLFFAGASDAAIVQGLVALLLRVYSGRPKSEILGTSPVFLHDMGLVKALSTNRGNGVEAMAQAIQKRASH

(19) United States
(12) Patent Application Publication
(10) Pub. No.: US 2011/0262976 A1
(43) Pub. Date: Oct. 27, 2011

(54) PRODUCTION OF R-a-LIPOIC ACID BY FERMENTATION USING GENETICALLY ENGINEERED MICROORGANISMS

(75) Inventors: Mahesh Kandula, Andhra Pradesh (IN); Mary E. Vaman Rao, Hopkinton, MA (US)

(73) Assignee: Indigene Pharmaceuticals, Inc., Westborough, MA (US)

(21) Appl. No.: 12/863,349

(22) PCT Filed: Jan. 16, 2009
(86) PCT No.: PCT/US09/00277

§ 371 Date: Jun. 24, 2011

Related U.S. Application Data
(62) Division of application No. 61/011,440, filed on Jan. 17, 2008.

(51) Int. Cl. C12P 17/00; C12N 15/74; C12N 1/21

(52) U.S. Cl. 435/117; 435/252.3; 435/320.1

(57) ABSTRACT

This application provides systems and methods for the production of R-α-lipoic acid. Lipoic acid synthesis genes may be expressed in an acid-tolerant microorganism, such as Gluconobacter oxydans. The lipoic acid synthesis proteins may include LipA and SufE. The genetically engineered strain may be cultured under suitable culture conditions, such as in a mannitol medium with an acidic pH.

### RELATED APPLICATIONS

This application claims the benefit of U.S. Provisional Application No. 61/011,440, filed Jan. 17, 2008, the specification of which is hereby incorporated herein by reference in its entirety.

### BACKGROUND

Alpha-lipoic acid is found in low quantities in a wide variety of microorganisms, as well as in plants and animals. It is a powerful antioxidant and has the ability to regenerate other natural antioxidants, such as glutathione, vitamin-C, vitamin-E, ubiquinone, and thioredoxin. It is a powerful free radical scavenging agent, acting on at least reactive oxygen species and reactive nitrogen species. Both forms of alpha lipoic acid (i.e. oxidized and reduced) have antioxidant abilities against oxidative stress-induced processes (Free Radical Biology & Medicine. Vol. 19, No. 2, 227-250, 1995 and Toxicology and Applied Pharmacology 182, 84-90, 2002).

R-alpha lipoic acid is an essential cofactor of particular multienzyme complexes in prokaryotes and eukaryotes. It is bound to a lysine residue of the respective enzyme to form a “lipoamide”. R-alpha lipoic acid may be covalently linked to the E2 subunit of, for example, pyruvate dehydrogenase or alpha keto dehydrogenase complexes, and plays an important role in redox transfer and as an acyl group donor in oxidative decarboxylation of alpha-ketoacids. R-alpha lipoic acid acts as an aminomethyl carrier in glycine cleavage enzyme systems. For a review refer: Proc. Natl. Acad. Sci. USA 97: 12481-6, 2000.

A number of synthetic methods for producing R-a-lipoic acid are available. However, the synthetic methodologies that are available are not economical, and are reported to have poor yields. In addition, synthetic methods tend to produce a racemic product (that is, equal amounts of the desired R product and the unwanted S product). Separation and purification of the desired enantiomer is difficult and costly. Thus, there is a need in the art for new methods of producing pure R-α-lipoic acid.

### SUMMARY OF THE INVENTION

Systems and methods for producing lipoic acid are described herein. Many organisms naturally produce low amounts of lipoic acid using endogenous biosynthetic pathways. These pathways produce pure R-α-lipoic acid, in contrast to chemical methods which produce a mixture of R-α-lipoic acid and S-α-lipoic acid. Applicants describe a method of overexpressing lipoic acid synthesis genes in a microorganism. These genes may include lipA, lipB, lplA, and Fe—S cluster genes like sufE. The microorganism may be an acid-tolerant microorganism. In a preferred embodiment, lipoic acid biosynthetic enzymes are localized to the periplasmic space so that lipoic acid synthesis takes place extracellularly. This allows one to collect lipoic acid from the medium without lysing the cells.

In certain embodiments, this application provides nucleic acids that may be used to produce lipoic acid. These nucleic acids may be used to express lipoic acid in otherwise wild-type microorganisms, or in mutant microorganisms.

In certain embodiments, this application provides nucleic acids for producing lipoic acid that are optimized for expression in an acidic medium. In certain embodiments, the nucleic acids are optimized for high expression. This optimization may involve use of a strong promoter. The optimization may involve use of a strong translation initiation sequence. The optimization may be codon optimization. Codon optimization may be used in producing a gene that intended for high expression in a given microorganism, including an acid-tolerant microorganism. Acid-tolerant microorganisms may include microorganisms of the genus Gluconobacter. Acid-tolerant microorganisms may include Gluconobacter oxydans, Gluconobacter suboxydans, Gluconobacter melanogenus, Gluconobacter albidus, Gluconobacter capsulatus, Gluconobacter cerinus, Gluconobacter dioxyacetonicus, Gluconobacter gluconicus, Gluconobacter industrius, or Gluconobacter nonoxygluconicus. Suitable microorganisms also include yeasts such as yeasts of the genus Saccharomyces, including Saccharomyces cerevisiae.

In certain embodiments, the isolated nucleic acid promotes production of lipoic acid. The nucleic acid may be a lipoic acid synthesis gene. The lipoic acid synthesis gene may be lipA, lipB, lplA, or an Fe—S cluster assembly protein. In certain embodiments, the isolated nucleic acid comprises a sequence that is at least 70%, 80%, 90%, 95%, 97%, 98%, 99%, 99.5%, or 100% identical to the nucleic acid of any of SEQ ID Nos. 1-6.

This application further provides for an isolated nucleic acid, comprising a sequence that hybridizes under stringent conditions to the nucleic acid of any of SEQ ID Nos. 1-6. Said nucleic acid may encode a protein with activity that is at least 25%, 50%, 75%, 80%, 90%, 95%, or 100% or greater of wild-type activity. Wherein the nucleic acid has a sequence that hybridizes under stringent conditions to the nucleic acid of SEQ ID No. 4, the protein activity may be measured by its ability to convert a synthetic tetrapeptide substrate, containing an N(epsilon)-octanoyl lysine residue, corresponding in sequence to the lipoyl binding domain of the E2 subunit of pyruvate dehydrogenase at a rate at least 50% of that of wild-type LipA. Wherein the nucleic acid has a sequence that hybridizes under stringent conditions to the nucleic acid of SEQ ID No. 5, activity may be measured by ability to transfer an octanoyl group from octanoyl-ACP to apo-H protein at a rate at least 50% of that of wild-type LipB. Wherein the nucleic acid has a sequence that hybridizes under stringent conditions to the nucleic acid of SEQ ID No. 6, activity may be measured by ability to bind SufB. In certain embodiments, the nucleic acid encodes a protein that binds SufB with a dissociation constant no more than twice, 5 times, 10 times, 20 times, 50 times, or 100 times the value of the dissociation constant of SufB and wild-type SufE. In certain embodiments, the nucleic acid encodes a protein that binds SufB with a dissociation constant less than 90%, 75%, 50%, 25%, or 10% the value of the dissociation constant of SufB and wild-type SufE. In certain embodiments, the nucleic acid encodes a protein that binds Sum with a dissociation constant equal to the value of the dissociation constant of SufB and wild-type SufE.

This application also provides an isolated nucleic acid, comprising a sequence that hybridizes under stringent conditions to the nucleic acid of SEQ ID No. 4 and the nucleic acid encodes a protein able to convert a synthetic tetrapeptide substrate, containing an N(epsilon)-octanoyl lysine residue, corresponding in sequence to the lipoyl binding domain of the E2 subunit of pyruvate dehydrogenase at a rate at least 25%, 50%, 75%, or 100% or more of that of wild-type LipA. In addition, this application provides an isolated nucleic acid, comprising a sequence that hybridizes under stringent conditions to the nucleic acid of SEQ ID No. 5 and the nucleic acid encodes a protein that transfers an octanoyl group from octanoyl-ACP to apo-H protein at a rate at least 25%, 50%, 75%, or 100% or more of that of wild-type LipB. This application further provides an isolated nucleic acid, comprising a sequence that hybridizes under stringent conditions to the nucleic acid of SEQ ID No. 6 and the nucleic acid encodes a protein that binds SufB with a dissociation constant no more than 1/2, 1 time, twice, four times, or 10 times the value of the dissociation constant of SufB and wild-type SufE.

In certain embodiments, at least one of said nucleic acid sequences is operably linked to a promoter. The promoter may be a heterologous promoter. The promoter may be an endogenous promoter. The promoter may be a constitutive promoter. The promoter may be selected from the group comprising rRNAB PI, P (from bacteriophage lambda), Pₐₙₜ (from bacteriophage P22), P_{spc}, P_{bla}, P1, P2, T3, T7, tufB, and any Gluconobacter ribosomal gene promoter. In certain embodiments, the promoter is an inducible promoter. In certain embodiments, an inducible promoter causes expression even in the absence of an inducer. This “leaky” expression is sometimes substantial, such that an inducer is not necessary to get satisfactory expression from the promoter. The promoter may be selected from the group consisting of pTrp, pTrc, pTac, PL, PT7, pBAD, and pLac. The promoter may be a repressible promoter. The promoter may promote overexpression. The promoter may also be any yeast promoter known in the art. Regulatable (i.e. inducible or repressible) promoters include GAL, GAL7, GAL10, CUP, ADH2, HSP30, MET25, MEL1, and PHO5. Constituitive promoters include ADH1, END1, GAP491, PGK1, MFα, MFα1, PYK1, and TP11.

In certain embodiments, the nucleic acid sequence or sequences may be operably linked to a nucleic acid tag. The tag may be a stabilization tag. The tag may be a tag used to assay protein levels. The tag may be a purification tag. The tag may be a localization tag. The localization tag may direct localization of a gene product to at least one of the periplasmic space, cell membrane, outer membrane, and the cell wall. The tag may comprise a nucleic acid sequence that is 70%, 80%, 90%, 95%, 97%, 99%, or 100% identical to a nucleic acid sequence of at least 20, 30, 40, 50, or 60 nucleotides selected from the 5′ or 3′ end of the nucleic acid of SEQ ID No. 7, SEQ ID No. 8, or SEQ ID No. 9. In addition, the tag may comprise a nucleic acid sequence that is 70%, 80%, 90%, 95%, 97%, 99%, or 100% identical to a nucleic acid sequence of at least 20, 30, 40, 50, or 60 nucleotides selected from the 5′ or 3′ end of the nucleic acid of any protein that is localized to the desired region of the cell.

Sequence tags in yeast include specific secretion signal peptides. Specific secretion signal peptides include MEL1, MFα1, PHO5, STA2, SUC2, AMY1, BLA, IFN, and GLU1. Tags that direct protein localization to the surface of a yeast cell are well known in the art.

For example, signal sequences that are recognized by SRP (the signal recognition particle) may direct protein localization to the surface of a cell.

The nucleic acid may be, for example, DNA, RNA, a PNA, a morpholino, single stranded, double stranded, methylated, and/or histone-associated.

This application additionally provides a fusion protein encoded by any of the nucleic acid sequences disclosed herein, such as a nucleic acid comprising a nucleic acid tag operably linked to the nucleic acid sequence of SEQ ID No. 4, SEQ ID No. 5, or SEQ ID No. 6 (or sequences 70%, 805, 90%, 95%, 97%, 98%, 99%, or 100% identical to these sequences). The tag may be, for instance, any of the tags listed herein, e.g. stabilization tags, tags used to assay protein levels, and purification tags. In some embodiments, the tag comprises a nucleic sequence at least 95% identical to at least 40 nucleotides selected from the N- or C-terminus of the nucleic acid of SEQ ID No. 7, SEQ ID No. 8, or SEQ ID No. 9.

This application further provides a vector for producing lipoic acid in a microorganism, comprising a sequence that is at least 95%, 97%, 99%, or 100% identical to SEQ ID No. 4. This application further provides a vector for producing lipoic acid in a microorganism, comprising a sequence that is at least 95%, 97%, 99%, or 100% identical to SEQ ID No. 5. This application further provides a vector for producing lipoic acid in a microorganism, comprising a sequence that is at least 95%, 97%, 99%, or 100% identical to SEQ ID No. 6. This disclosure also provides a vector for producing lipoic acid in a microorganism, comprising a nucleic acid sequence hybridizes under stringent conditions to the nucleic acid of SEQ ID No. 4, and the nucleic acid encodes a protein able to convert a synthetic tetrapeptide substrate, containing an N(epsilon)-octanoyl lysine residue, corresponding in sequence to the lipoyl binding domain of the E2 subunit of pyruvate dehydrogenase at a rate at least 25%, 50%, 75%, or 100% or more of that of wild-type LipA. This disclosure also provides a vector for producing lipoic acid in a microorganism, comprising nucleic acid sequences encoding SufE (e.g. SEQ ID No. 3 or 6) and LipA (e.g. SEQ ID No. 1 or 4). In certain embodiments, the vector comprises a promoter that causes overexpression of one or both of SufE and LipA. In certain embodiments, the vector encodes one or more of the proteins in SEQ ID No. 10, 11, or 12, or a protein at least 70%, 80%, 90%, 95%; 97%, 98%, 99%, or 100% identical to it. In certain embodiments, the vector encodes an active fragment of one of said sequences.

The vector may further comprise an additional lipoic acid synthesis gene. The additional lipoic acid synthesis gene may be an Fe—S cluster assembly gene. The Fe—S cluster assembly gene may be sufE. The Fe—S cluster assembly gene may be selected from the group consisting of iscR, iscS, iscU, iscA, hscB, hscA, iscX, iscI, iscII, csd, sufA, sufB, sufC, sufD, sufE, sufS, nifS, nifU, and nfs1. The Fe—S cluster assembly gene may be any Fe—S cluster assembly gene known in the art, including Fe—S cluster assembly genes found in yeast.

The vector may comprise a backbone. The backbone may be based on any appropriate backbone, such as pUC57 or pCDF-Duet1. The vector may be capable of maintenance in one, two, or more hosts. The vector may comprise a selectable marker. The selectable marker may be any marker known in the art including a marker selected from the group consisting of ampR, emR, kanR, chlorR, smR, tetR, genR, leu+, ura+, trp+, his+, lys+, met+, and ade+. The selectable marker may be LEU, URA, TRP, HIS, LYS, MET, and/or ADE. The selectable marker may be LEU2, URA3, TRP1, HIS3, LYS2, MET17, and/or ADE2. The vector may comprise a transcription terminator. The vector may comprise a polyadenylation sequence. The vector may comprise at least one origin of replication. The origin of replication may be selected from the group consisting of OriV and the origins found on plasmids pACYC184, RP4, RSF1010, pBR322, pACYC177, pACYC184, pSC101, pGE1, pGE2, pGO32935, pSUP301, pVK102, pGOX1, pGOX2, pGOX3, pGOX4, pGOX5, pMB1, and the origins found on bacteriophages lambda, P1, and T-coliphages. The origin of replication may be any origin known to function in yeast. The origin may include an ARS sequence.

This application further provides a vector for producing lipoic acid in a microorganism, comprising a nucleic acid sequence that hybridizes under stringent conditions to the nucleic acid of SEQ ID No. 4. The nucleic acid may encode a protein with activity that is at least 25%, 50%, 75%, 80%, 90%, 95%, or 100% or greater of wild-type activity.

The disclosures herein provide an acid-tolerant microorganism comprising a nucleic acid that is at least 70%, 805, 90%, 95%, 97%, 98%, 99%, or 100% identical to the nucleic acid of SEQ ID No. 4, SEQ ID No. 5, or SEQ ID No. 6. At least one of said nucleic acid sequences may be overexpressed. At least one of the nucleic acid sequences may be in a vector. The vector may be any appropriate vector described in this document. The instant disclosures also describe an acid-tolerant microorganism comprising a nucleic acid sequence that that hybridizes under stringent conditions to the nucleic acid of SEQ ID No. 4, SEQ ID No. 5, or SEQ ID No. 6, and the nucleic acid encodes a protein with at least some activity. Herein is also provided an acid-tolerant microorganism comprising a nucleic acid sequence that that hybridizes under stringent conditions to the nucleic acid of SEQ ID No. 4 and the nucleic acid encodes a protein able to convert a synthetic tetrapeptide substrate, containing an N(epsilon)-octanoyl lysine residue, corresponding in sequence to the lipoyl binding domain of the E2 subunit of pyruvate dehydrogenase at a rate at least 25%, 50%, 75%, or 100% or more of that of wild-type LipA. This disclosure also provides an acid-tolerant microorganism comprising a nucleic acid sequence that that hybridizes under stringent conditions to the nucleic acid of SEQ ID No. 5, and the nucleic acid encodes a protein that transfers an octanoyl group from octanoyl-ACP to apo-H protein at a rate at least 25%, 50%, 75%, or 100% or more of that of wild-type LipB. Furthermore, this application provides an acid-tolerant microorganism comprising a nucleic acid sequence that that hybridizes under stringent conditions to the nucleic acid of SEQ ID No. 6 and the nucleic acid encodes a protein that binds SufB with a dissociation constant no more than twice the value of the dissociation constant of SufB and wild-type SufE. This application also provides an acid-tolerant microorganism that overexpresses SufE (e.g. SEQ ID No. 3 or 6) and LipA (e.g. SEQ ID No. 1 or 4). In certain embodiments, the microorganism expresses one or more of the proteins in SEQ ID No. 10, 11, or 12, or a protein at least 70%, 80%, 90%, 95%, 97%, 98%, 99%, or 100% identical to it. In certain embodiments, the microorganism expresses an active fragment of one of said sequences.

In the microorganism, in some embodiments, at least one of said nucleic acid sequences is overexpressed. At least one of said nucleic acid sequences may be in a vector. The vector may comprise any of the components listed herein, such as an additional lipoic acid synthesis gene (e.g. sufE, iscR, iscS, iscU, iscA, hscB, hscA, iscX, iscI, iscII, csd, sufA, sufB, sufC, sufD, sufE, sufS, nifS, nifU, and nfs1), a selectable marker (e.g. ampR, emR, kanR, chlorR, smR, tetR, genR, leu+, ura+, trp+, his+, lys+, met+, and ade+), a transcription terminator, an origin of replication (e.g. OriV and the origins found on plasmids pACYC184, RP4, RSF1010, pBR322, pACYC 177, pACYC184, pSC101, pGE1, pGE2, pGO32935, pSUP301, pVK102, pMB1, and the origins found on bacteriophages lambda, P1, and T-coliphages), and a promoter (e.g. rRNAB P1, P (from bacteriophage lambda), Pₐₙₜ (from bacteriophage P22), P_{spc}. P_{bla}, P 1, P2, T3, T7, tufB, any Gluconobacter ribosomal gene promoter, pTrp, pTrc, pTac, PL, PT7, pBAD, and pLac). In the microorganism, at least one protein encoded by said nucleic acid may be localized to at least one of the cell membrane, outer membrane, periplasmic space, cell wall, the cytoplasm, the mitochondria, the nucleus, and the endoplasmic reticulum of said microorganism. The microorganism may further overexpress at least one of ACP and an E2 domain protein. E2 domain proteins include proteins in the pyruvate dehydrogenase complex and proteins in the alpha keto dehydrogenase complexes.

The nucleic acid may be integrated into the genome of the microorganism. The nucleic acid may be present on a vector including a plasmid and a shuttle vector. The nucleic acid may be present in multiple copies in the microorganism. At least one of said nucleic acid sequences may be overexpressed.

In certain embodiments, the microorganism is a bacterium of the genus Gluconobacter. The microorganism may be Gluconobacter oxydans. The microorganism may be of the species Gluconobacter suboxydans, Gluconobacter melanogenus, Gluconobacter albidus, Gluconobacter capsulatus, Gluconobacter cerinus, Gluconobacter dioxyacetonicus, Gluconobacter gluconicus, Gluconobacter industrius, or Gluconobacter nonoxygluconicus. In other embodiments, the microorganism is a species of yeast, such as a species of the genus Saccharomyces (like Saccharomyces cerevisiae).

This application additionally provides methods of producing lipoic acid. Such a method may comprise culturing an acid-tolerant microorganism comprising a nucleic acid sequence that is at least 95%, 96%, 97%, 98%, 99%, or 100% identical to the nucleic acid of SEQ ID No. 4, SEQ ID No. 5, or SEQ ID No. 6 in a culture medium and isolating lipoic acid from the culture medium. This disclosure additionally provides a method of producing lipoic acid, comprising culturing the an acid-tolerant microorganism comprising a nucleic acid sequence that hybridizes under stringent conditions to the nucleic acid of SEQ ID No. 4, and the nucleic acid encodes a protein able to convert a synthetic tetrapeptide substrate, containing an N(epsilon)-octanoyl lysine residue, corresponding in sequence to the lipoyl binding domain of the E2 subunit of pyruvate dehydrogenase at a rate at least 25%, 50%, 75%, or 100% or more of that of wild-type LipA. This disclosure also provides a method of producing lipoic acid, comprising culturing the an acid-tolerant microorganism comprising a nucleic acid sequence that hybridizes under stringent conditions to the nucleic acid of SEQ ID No. 5 and the nucleic acid encodes a protein that transfers an octanoyl group from octanoyl-ACP to apo-H protein at a rate at least 25%, 50%, 75%, or 100% or more of that of wild-type LipB. Still further, this application provides a method of producing lipoic acid, comprising culturing the an acid-tolerant microorganism comprising a nucleic acid sequence that hybridizes under stringent conditions to the nucleic acid of SEQ ID No. 6 and the nucleic acid encodes a protein that binds SufB with a dissociation constant no more than 1/2, 1 time, twice, four times, or 10 times the value of the dissociation constant of SufB and wild-type SufE. Such methods may comprise culturing any appropriate microorganism described herein in a culture medium and isolating lipoic acid from the culture medium.

The microorganism may be any of those discussed herein, e.g. bacterium of the genus Gluconobacter (such as Gluconobacter oxydans) or a species of yeast (e.g. Saccharomyces cerevisiae)

In certain embodiments of the methods herein, one of said nucleic acid sequences is overexpressed. In addition, at least one of said nucleic acid sequences may be in a vector such as those described above. (For example, the vector may comprise at least one of an additional lipoic acid synthesis gene, a selectable marker, a transcription terminator, an origin of replication, and a promoter, examples of which are provided above). In the methods, at least one protein encoded by said nucleic acid may be localized to at least one of the cell membrane, outer membrane, periplasmic space, cell wall, the cytoplasm, the mitochondria, the nucleus, and the endoplasmic reticulum of said microorganism. The microorganism may further overexpress at least one of ACP and an E2 domain protein. The nucleic acids may be integrated, in single copy, or in multiple copies.

The instant application provides various methods of producing lipoic acid. Some such methods include a step of (a) providing an acid-tolerant microorganism that overexpresses lipB and at least one of lipA and an Fe—S cluster assembly gene. These methods may include a step of (b) culturing said bacterium in culture medium. These methods may include a step of (c) isolating lipoic acid from the culture medium.

At least one of the genes of step (a) may be present in multiple copies in the microorganism. In some embodiments, at least one of the genes of step (a) is operably linked to a promoter. The promoter may be any suitable promoter, including any of those listed in this application. One of the nucleic acid sequences may be operably linked to a nucleic acid tag, including any of the tags listed herein.

The instant application provides, inter alia, method of producing lipoic acid, comprising culturing the an acid-tolerant microorganism comprising a nucleic acid sequence that hybridizes under stringent conditions to the nucleic acid of SEQ ID No. 4, SEQ ID No. 5, or SEQ ID No. 6, and the nucleic acid encodes a protein with at least some activity, in a culture medium and isolating lipoic acid from the culture medium.

This application additionally provides a method of producing lipoic acid, comprising culturing an acid-tolerant microorganism comprising a fusion protein, wherein the fusion protein comprises a polypeptide tag operably linked to LipA, LipB, or an Fe—S cluster assembly protein in a culture medium and isolating lipoic acid from the culture medium. The polypeptide tag may be any of those listed herein.

Still further, this application provides a method of producing lipoic acid, comprising culturing an acid-tolerant microorganism comprising a SufE (e.g. SEQ ID No. 3 or 6) and a LipA (e.g. SEQ ID No. 1 or 4) gene. In some embodiments, the LipA and SufE genes are on the same vector or on two different vectors. In some embodiments, one or both of the LipA and SufE genes is overexpressed.

The microorganism may overexpress both lipA and an Fe—S cluster gene. The Fe—S cluster assembly gene may be, for example, sufE, iscR, iscS, iscU, iscA, hscB, hscA, iscX, iscI, iscII, csd, sufA, sufB, sufC, sufD, sufE, sufS, nifS, nifU, or nfs1. More than one Fe—S cluster assembly gene may be overexpressed.

In certain embodiments, the medium further comprises an agent that induces gene expression. The agent may be selected from the group consisting of octanoic acid, tetracycline, galactose, IAA, IPTG, arabinose, and nalidixic acid. The medium may further comprise an agent that represses gene expression. The agent may be selected from the group consisting of tetracycline, galactose, and tryptophan.

The medium may further comprise a precursor of lipoic acid. The precursor may be, for example, octanoic acid, octanoate, or an octanoylated molucule such as octanoyl-AMP. The medium may also include the following: fatty acid synthesis pathway intermediates, octanoic esters or caprylic aldehyde or alcohol or any substrate or any metabolite or any chemical solvent or any carbohydrate. Such molecules may be precursors or intermediates for lipoic acid synthesis.

In some embodiments, the medium further comprises an antibiotic. The antibiotic may be ampicillin, penicillin, kanamycin, tetracycline, streptomycin, erythromycin, chloramphenicol, gentamicin, or any combination thereof.

In some aspects, the microorganism is cultured in a shaker flask. In certain embodiments, said microorganism is cultured at between 25° C. and 30° C., for instance at about 26° C. In certain embodiments, said microorganism is cultured at between 150 and 700 rpm, for example at between 150 and 250 rpm, such as 170 rpm. In certain embodiments, the lipoic acid is isolated from the culture medium, for instance using HPLC as described in Example 4.

In certain embodiments, the resulting lipoic acid has less than 50%, 25%, 10%, 5%, 2%, 1%, 0.5%, or 0.1% of one or more impurities. Impurities may be any undesired substance such as nucleic acids, proteins, small organic molecules, minerals, lipids, carbohydrates, and salts. In certain embodiments, the resulting lipoic acid has less than 1% oligomer content. In certain embodiments, the resulting lipoic acid is crystallized. In certain embodiments, both lipoic acid and a second molecule are isolated from said culture.

In certain embodiments, the microorganism is first cultured in a medium containing an antibiotic and the bacterium is then transferred to a medium where that antibiotic is present at a reduced level or the antibiotic is absent. In certain embodiments, the microorganism is first cultured in a medium with no antibiotic or low levels of an antibiotic and the bacterium is then transferred to a medium where an antibiotic is present or present at a higher level. In certain embodiments, the lipoic acid isolated comprises R-lipoic acid and S-lipoic acid. In certain embodiments, the amount of R-lipoic acid is greater than the amount of S-lipoic acid. For example, R-lipoic acid may make up at least 60%, 70%, 80%, 90%, 95%, 97%, 98%, 99%, 99.5%, or 100% of the lipoic acid. In certain embodiments, the amount of S-lipoic acid is greater than the amount of R-lipoic acid. In certain preferred embodiments, the lipoic acid isolated is R-lipoic acid and is essentially free of S-lipoic acid.

In any of the disclosed methods, the microorganism may be of a genus selected from the group consisting of Lactobacillus, Acetobacter, Azotobacter, Bacillus, Ervinia, and Thiobacillus. In addition, the microorganism may be selected from the group consisting of Acetobacter aceti, Bacillus megaterium, Ervinia carotovora, and Thiobacillus ferrooxidans. In addition, the microorganism may be of the genus Gluconobacter. In addition, the microorganism may be Gluconobacter oxydans, Gluconobacter suboxydans, Gluconobacter melanogenus, Gluconobacter albidus, Gluconobacter capsulatus, Gluconobacter cerinus, Gluconobacter dioxyacetonicus, Gluconobacter gluconicus, Gluconobacter industrius, or Gluconobacter nonoxygluconicus. In addition, the microorganism may be yeasts such as yeasts of the genus Saccharomyces, including Saccharomyces cerevisiae. The microorganism may be any of the microorganisms disclosed in this application or any of the references recited herein.

In certain embodiments, the genes (e.g. the genes of step (a)) are codon-optimized for expression in the acid-tolerant microorganism. The genes may be codon-optimized for expression in Gluconobacter. The codon-optimized genes may be about 80% identical to the corresponding wild-type genes. The codon-optimized genes may alternatively be between 30% and 99% identical to the corresponding wild-type genes, e.g. greater than 30%, 40%, 50%, or 60%, or less than 99%, 95%, 80%, or 70%. The genes of step (a) may be integrated into the host genome or present on one or more vectors. For instance, a microorganism may express SEQ ID No. 4 or a mutant thereof on one vector, and SEQ ID No. 6 or a mutant thereof on a second vector. In some aspects, the microorganism further overexpresses at least one of ACP and an E2 domain protein.

The disclosures herein contemplate, inter alia, a fusion protein comprising a polypeptide tag operably linked to a lipoic acid synthesis protein. The lipoic acid synthesis protein may be LipA, LipB, LplA, or an Fe—S cluster assembly protein (including any of the Fe—S cluster assembly proteins listed herein). The tag may be any tag listed herein, including a localization tag, a tag used to assay protein levels, or a purification tag. Any nucleic acid encoding such a fusion protein is also contemplated within the scope of the disclosure. The nucleic acid may be codon-optimized. The nucleic acid may be part of any vector laid out in this disclosure. The nucleic acid may be integrated into the genome. The nucleic acid may be present on a vector, such as a shuttle vector. The nucleic acid may be present in a single copy or in multiple copies. The nucleic acid may also have a promoter, selectable marker, or origin of replication, examples of which may be found herein. The nucleic acid may also have a transcription terminator or polyadenylation sequence. In certain embodiments, the vector encodes at least two, three, four, or more fusion proteins, each fusion protein comprising a polypeptide tag operably linked to LipA, LipB, or an Fe—S cluster assembly protein.

The tag may be selected from the group consisting of stabilization tags, tags used to assay protein levels, purification tags, and localization tags. The tag may also improve solubility of the protein. The tag may prevent unwanted protein-protein interactions. The tag may direct localization of a gene product to at least one of the periplasmic space, cell membrane, outer membrane, cell wall, nucleus, endoplasmic reticulum, mitochondria, Golgi apparatus, and cytoplasm.

The present application also provides an acid-tolerant microorganism, comprising any fusion protein described herein. Specifically, the fusion protein may comprise a polypeptide tag operably linked to LipA, LipB, or an Fe—S cluster assembly protein. The microorganism may overexpress the fusion protein. The microorganism may comprise any nucleic acid necessary for encoding said fusion protein, e.g. the vector described herein. The microorganism may be any one of those disclosed herein, for example a species of yeast or a bacterium of the genus Gluconobacter.

In certain embodiments, at least one one of said fusion proteins is localized to at least one of the cytoplasm, the mitochondria, the nucleus, and the endoplasmic reticulum of said microorganism.

The instant application also provides methods of producing lipoic acid. Such methods may include a step of culturing an acid-tolerant microorganism comprising a fusion protein. The fusion protein may comprise a polypeptide tag operably linked to LipA, LipB, or an Fe—S cluster assembly protein. The microorganism may be cultured in a culture medium. One may further isolate lipoic acid from the culture medium.

All the claims of the instant application are hereby appended to this section.

Before the present systems and methods herein are further described, it is to be understood that these systems and methods are not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosures. The upper and lower limits of these smaller ranges may independently be included in the smaller ranges, and are also encompassed within the disclosures, subject to any specifically excluded limit in the stated range. Where the stated range includes one or both of the limits, ranges excluding either or both of those included limits are also included in the disclosures.

Unless defined otherwise, all technical and scientific terms used herein have the same meaning as commonly understood by one of ordinary skill in the art. Although any methods and materials similar or equivalent to those described herein can also be used in the practice or testing of the present systems and methods, select preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates which may need to be independently confirmed.

### BRIEF DESCRIPTION OF THE DRAWINGS

FIG. 1 is a table depicting the preferred codon usage in Gluconobacter oxydans.

FIG. 2 is a map of a plasmid with a pUC57 backbone and a LipA insert and an ampicillin resistance marker.

FIG. 3 is a map of a plasmid with a pUC57 backbone and a LipB insert and an ampicillin resistance marker.

FIG. 4 is a map of a plasmid with a pUC57 backbone and a SufE insert and an ampicillin resistance marker.

FIG. 5 is a map of a plasmid with both LipA and LipB inserts, and ampicillin and gentamicin resistance markers.

FIG. 6 is a map of a plasmid with a LipA insert and a gentamicin resistance marker.

FIG. 7 is a map of a plasmid with a SufE insert and an ampicillin resistance marker.

### DETAILED DESCRIPTION

### Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “acid-tolerant microorganism” is used herein to refer to a microorganism that may be stably cultured in acidic conditions. For example, an acid-tolerant microorganism may be stably cultured at a pH below 6, 5, 4, 3, or 2. As used herein, acid-tolerant microorganisms include both wild-type and genetically modified microorganisms. In addition, a microorganism that is not acid-tolerant may be genetically modified to become acid tolerant. For example, artificial selection (alone or combined with random mutagenesis) may be used to increase acid tolerance. In addition, targeted genetic modifications may be used to increase acid tolerance in a microorganism. For example, transformation with transmembrane pump genes, buffer proteins, or buffer synthesis enzymes may be used to increase the acid tolerance of a microorganism.

The term “codon optimization” refers to the process of making silent mutations in a gene, to substitute one codon for another while encoding the same amino acid sequence. The new codon should be a codon that permits high level expression in a given organism. The new codon is often the codon that is most frequently used to encode a given amino acid in that organism. A gene thus modified is considered “codon-optimized”.

By “construct” is meant a recombinant nucleic acid, generally recombinant DNA, which has been generated for the purpose of the expression of a specific nucleotide sequence(s), or is to be used in the construction of other recombinant nucleotide sequences. A construct may be a vector. A construct may also be, for example, a YAC, BAC, shuttle vector, or cosmid.

The verb “culture” is used herein to refer to incubating a cell, such as a microorganism, in an appropriate medium. Culturing may include maintaining the cell in conditions that stimulate growth. Culture conditions may include a specific temperature, a specific amount of gasses such as oxygen or carbon dioxide, a specific amount of shaking or orbital rotation, and a specific amount of light. “Stably culture” refers to the ability to culture a cell for the long term. A stable culture is, for example, a culture that permits the cell to survive for a period of 2, 4, 6, 8, 10, or more days. A stable culture is also, for example, a culture that permits the cell to divide for a period of 2, 4, 6, 8, 10, or more days.

As used herein, the term “exogenous nucleic acid” refers to a nucleic acid that is not normally or naturally found in and/or produced by a given bacterium, organism, or cell in nature. As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell. The “endogenous promoter” of a gene is the DNA sequence that controls expression of that gene by recruiting RNA polymerase, transcription factors, or other transcriptional machinery.

The term “expressing” refers to the process where a host cell transcribes a gene. The gene may be exogenous or endogenous. The gene may also be translated, and may produce an active protein product. The term “expressing” also includes “overexpressing” and “underexpressing”.

“Fe—S cluster assembly proteins” are proteins that promote the formation of Fe—S clusters in substrate proteins. Fe—S clusters are iron-sulfur clusters containing sulfide-linked di-, tri-, and tetrairon centers in variable oxidation states. Fe—S cluster assembly genes are genes that encode Fe—S cluster assembly proteins. Examples of Fe—S cluster assembly proteins include IscR, IscS, IscU, IscA, HscB, HscA, IscX, IscI, IscII, Csd, SufA, SufB, SufC, SufD, SufE, SufS, NifS, NifU, and Nfs1.

As used herein, the term “gene product” refers to RNA encoded by DNA (or vice versa) or protein that is encoded by an RNA or DNA, where a gene will typically comprise one or more nucleotide sequences that encode a protein, and may also include 5′ and 3′ untranslated regions or other non-coding nucleotide sequences.

The term “heterologous nucleic acid,” as used herein, refers to a nucleic acid wherein at least one of the following is true: (a) the nucleic acid is foreign (“exogenous”) to (i.e., not naturally found in) a given host microorganism or host cell; (b) the nucleic acid comprises a nucleotide sequence that is naturally found in (e.g., is “endogenous to”) a given host microorganism or host cell (e.g., the nucleic acid comprises a nucleotide sequence endogenous to the host microorganism or host cell); however, in the context of a heterologous nucleic acid, the same nucleotide sequence as found endogenously is produced in an unnatural (e.g., greater than expected or greater than naturally found) amount in the cell, or a nucleic acid comprising a nucleotide sequence that differs in sequence from the endogenous nucleotide sequence but encodes the same protein (having the same or substantially the same amino acid sequence) as found endogenously is produced in an unnatural (e.g., greater than expected or greater than naturally found) amount in the cell; (c) the nucleic acid comprises two or more nucleotide sequences that are not found in the same relationship to each other in nature, e.g., the nucleic acid is recombinant. An example of a heterologous nucleic acid is a nucleotide sequence encoding a lipoic acid synthesis gene operably linked to a transcriptional control element (e.g., a promoter) to which an endogenous (naturally-occurring) a lipoic acid synthesis coding sequence is not normally operably linked. Another example of a heterologous nucleic acid is a high copy number plasmid comprising a nucleotide sequence encoding a lipoic acid synthesis protein. A heterologous promoter may be a promoter that drives expression of a gene, wherein the heterologous promoter is different from the promoter that drives expression of the same gene (or homolog or variant thereof) in a wild-type organism.

A “host cell,” as used herein, denotes a cultured cell (e.g. eukaryotic or prokaryotic), which cell can be, or has been, used as a recipient for a nucleic acid (e.g., an expression vector that comprises a nucleotide sequence encoding one or more biosynthetic pathway gene products such as lipoic acid synthesis gene products), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject prokaryotic host cell is a genetically modified prokaryotic host cell (e.g., a bacterium), by virtue of introduction into a suitable prokaryotic host cell a heterologous nucleic acid, e.g., an exogenous nucleic acid that is foreign to (not normally found in nature in) the prokaryotic host cell, or a recombinant nucleic acid that is not normally found in the prokaryotic host cell; and a subject eukaryotic host cell is a genetically modified eukaryotic host cell, by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

The term “including” is used to mean “including but not limited to”. “Including” and “including but not limited to” are used interchangeably.

An “inducible promoter” is a promoter that allows no transcription or low-level transcription in the absence of an inducer. Upon addition of an agent that induces gene expression (“inducer”), the gene transcription is increased. A “repressible promoter” is a promoter that allows transcription in the absence of a repressor. Upon addition of the agent that represses gene expression (“repressor”), the gene transcription is decreased or completely abolished.

The term “isolate” refers to the process of substantially separating a desired composition of matter from a mixture or solution. Isolation may include removing 50%, 75%, 90%, 95%, 99%, or essentially 100% of an unwanted component. With reference to isolated lipoic acid, isolated lipoic acid includes lipoic acid that is free (e.g. not bound to a protein.) Isolated lipoic acid may refer to isolated R-alpha lipoic acid, lacking S-alpha lipoic acid. As used herein regarding nucleic acids of polypeptides, the term “isolated” is meant to describe a polynucleotide or a polypeptide that is in an environment different from that in which the polynucleotide or polypeptide naturally occurs.

As used herein, the term “lipoic acid” also encompasses its conjugate base, lipoate. The term “lipoic acid” also includes both stereoisomers (the R and S forms) of lipoic acid and lipoate, as well as all the particular salts of the lipoic acid, such as, for example, the calcium, potassium, magnesium, sodium, or ammonium salt. The term “lipoic acid” also encompasses lipoic acid in its free form as well as in a form bound to ACP, AMP, an E2 domain, or other molecules.

The active, R form of lipoic acid may be referred to by several synonymous terms: R-lipoic acid, R-alpha lipoic acid, R-α-lipoic acid, R—configuration of alpha lipoic acid, and R-(+)-alpha lipoic acid, as well as any other term understood in the art to refer to the R form of lipoic acid.

The term “lipoic acid synthesis gene” refers to genes that participate in the synthesis of lipoic acid directly or indirectly. Such genes include genes that when knocked out cause an organism to produce less lipoic acid than a corresponding wild-type organism. Specific examples of lipoic acid synthesis genes include lipA, lipB, lplA, and Fe—S cluster genes including sufE. Lipoic acid synthesis proteins are the proteins produced by lipoic acid synthesis genes. As used herein, “lipoic acid synthesis gene” and “lipoic acid synthesis protein” include both wild-type and mutant versions. A specific mutant version included is one in which the gene is fused to a heterologous sequence. Substitution and truncation mutants are also included. Silent mutations are included. Mutations that optimize codon usage are included. Any mutation that does not substantially change the activity of the protein is included. Mutations that increase or decrease the activity of the protein are also included. Mutations that increase the heat-resistance and/or acid-resistance of the protein are also included. The term “lipoic acid synthesis gene” also refers to any DNA sequence with at least 95% identity to a wild-type lipoic acid synthesis gene such SEQ ID Nos. 1-3. The term “lipoic acid synthesis gene” also refers to any DNA sequence with at least 95% identity to a codon-optimized lipoic acid synthesis gene such SEQ ID Nos. 4-6.

The term “metabolic pathway” refers to a series of two or more enzymatic reactions in which the product of one enzymatic reaction becomes the substrate for the next enzymatic reaction. At each step of a metabolic pathway, intermediate compounds are formed and utilized as substrates for a subsequent step. These compounds may be called “metabolic intermediates.” The products of each step are also called “metabolites.”

The term “microorganism” refers to any organism too small to be viewed by the unaided eye, as bacteria, protozoa, and some fungi and algae. Microorganisms include E. colt, S. cerevisiae, S. pombe, and Gluconobacter oxydans; other examples are listed throughout the specification.

The term “naturally-occurring” as used herein as applied to a nucleic acid, a cell, or an organism, refers to a nucleic acid, cell, or organism that is found in nature. For example, a polypeptide or polynucleotide sequence that is present in an organism (including viruses) that can be isolated from a source in nature and which has not been intentionally modified by a human in the laboratory is naturally occurring. As used herein, “naturally occurring” may be synonymous with “wild-type”.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature. In addition, a localization tag operably linked to a protein may direct the localization of that protein to a specific region of a cell.

The term “overexpress” is used to mean increasing the production of a gene and/or protein above endogenous levels. Overexpression may result in, for example, a 50%, 2-fold, 5-fold, 10-fold, or greater increase over endogenous levels. Overexpression may be accomplished, for example, by operably linking a strong promoter to the gene to be overexpressed.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403 10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173 187 (1997). Also, the GAP program using the Needleman and Wunsch alignment method can be utilized to align sequences. See J. Mol. Biol. 48: 443 453 (1970).

“Recombinant,” as used herein, means that a particular nucleic acid (DNA or RNA) is the product of various combinations of cloning, restriction, ligation, and/or in vitro DNA synthesis steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms.

Thus, e.g., the term “recombinant” polynucleotide or nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

The term “signal sequence” refers to a peptide sequence (or the nucleic acid encoding that sequence) that directs localization of a peptide to a specific location within the cell. Specifically, a signal sequence may direct a peptide to the periphery of the cell including the periplasmic space.

The term “silent mutation” refers to changes to a DNA sequence that do not affect the protein sequence it encodes.

“Synthetic nucleic acids” can be assembled from oligonucleotide building blocks that are chemically synthesized using procedures known to those skilled in the art. These building blocks are ligated and annealed to form gene segments which are then enzymatically assembled to construct the entire gene. “Chemically synthesized,” as related to a sequence of DNA, means that the component nucleotides were assembled in vitro. Manual chemical synthesis of DNA may be accomplished using well-established procedures, or automated chemical synthesis can be performed using one of a number of commercially available machines. The nucleotide sequence of the nucleic acids can be modified for optimal expression based on optimization of nucleotide sequence to reflect the codon bias of the host cell. The skilled artisan appreciates the likelihood of successful expression if codon usage is biased towards those codons favored by the host. Determination of preferred codons can be based on a survey of genes derived from the host cell where sequence information is available.

The term “tag ” may refer to a nucleic acid tag or a protein tag. The term “nucleic acid tag” refers to a nucleic acid sequence that may be fused to another nucleic acid sequence. The term “protein tag” or “polypeptide tag” refers to the polypeptide encoded by a nucleic acid tag. Preferably, a tag confers additional functionality on the gene product. For instance, a stabilization tag may increase the stability of a protein in a cell. It may do so by, for instance, by masking a degradation element in the protein. As another example, a tag that is used to assay protein levels may be a tag that can be detected by Western blot such as a FLAG, myc, or his tag. Alternatively, a fluorescent protein tag such as GFP may be used to assay protein levels with an optical assay. A purification tag is a tag that may be used to purify the protein to which the tag is operably linked. Purification tags include, for instance, FLAG, myc, his, TAP, and GST tags. Localization tags are any tag that directs the protein to which the tag is fused to a specific region of a cell. Localization tags include signal sequences, membrane-spanning peptides, nuclear localization sequences, and mitochondrial targeting sequences. Any tag known in the art may be used in accordance with the methods herein.

The term “transformation” refers to any method for introducing foreign molecules, such as DNA, into a cell. Transformation may result in a genetically modified organism. Lipofection, DEAE-dextran-mediated transfection, microinjection, protoplast fusion, calcium phosphate precipitation, retroviral delivery, electroporation, natural transformation, and biolistic transformation are just a few of the methods known to those skilled in the art which may be used. Transformation can be accomplished by incorporation of the new DNA into the genome of the host cell, or by transient or stable maintenance of the new DNA as an episomal element. Permanent changes can be introduced into the chromosome or via extrachromosomal elements such as plasmids and expression vectors, which may contain one or more selectable markers to aid in their maintenance in the recombinant host cell.

### Introduction

Gluconobacter oxydans is a gram-negative bacterium belonging to the family Acetobacteraceae. G. oxydans is an obligate aerobe, having a respiratory type of metabolism using oxygen as the terminal electron acceptor. Gluconobacter strains flourish in sugary niches e.g. ripe grapes, apples, dates, garden soil, baker's soil, honeybees, fruit, cider, beer, and wine. Gluconobacter strains are non-pathogenic towards man and other animals but are capable of causing bacterial rot of apples and pears accompanied by various shades of browning. Applicants describe herein, inter alfa, a method of producing lipoic acid using Gluconobacter.

Many wild-type organisms produce low levels of lipoic acid. There are at least two converging pathways by which a cell may make lipoic acid. A starting precursor may be extracellular octanoic acid. Octanoic acids enters the cell, and LplA (lipoyl-protein ligase) covalently links it to an E2 domain of a protein. Alternatively, the starting precursor octanlyl-ACP (acyl carrier protein) may be generated intracellularly through the fatty acid synthesis pathway. LipB (lipoyl transferase) covalently bonds octanoyl-ACP to an E2 protein domain. Thus, both pathways result in the same intermediate substrate: an octanoylated E2 domain. Next LipA (lipoic acid synthase) acts upon the octanoylated E2 domain by donating two sulfur atoms from L-cysteine to carbons 6 and 8 of the octanoyl group. The resulting product is a lipoylated E2 domain, which is an E2 domain conjugated to lipoic acid.

One of the novel disclosures of the present disclosure is that LipA can act on free octanoic acid (in addition to octanoic acid bound to an E2 domain). Structural data as well as in vitro enzymatic data support this notion. For instance, LipA performs catalysis on the end of the substrate that is farthest from the E2 domain. Thus, Applicants herein disclose that it should be possible to produce lipoic acid in its free form, without attachment to an E2 domain.

Most proteins can not pass easily through a cell membrane. Thus, the free form of lipoic acid is significantly more cell-permeable than the E2-bound form. A preferred method of producing lipoic acid, disclosed in this application, involves producing lipoic acid extracellularly. In another embodiment, free lipoic acid is produced within the host cell and is allowed to diffuse into the medium.

Considering the lipoic acid synthesis pathway from a different point of view, one may focus on only octanoic acid as a precursor. However, the octanoic acid may be endogenous or exogenous. If octanoic acid is endogenous, it may covalently bond with ACP in a spontaneous (enzyme-free) reaction. If octanoic is exogenous, it may covalently bond with ATP (to form octanoate-AMP) in a spontaneous reaction. Next, free radicals may also be generated in a spontaneous process. Finally, LipA donates sulfur atoms as described above.

When LipA donates sulfur atoms, LipA's Fe—S cluster is changed from Fe₄S₄ to Fe₄S₃. The Fe—S cluster is then replenished before the next catalytic reaction. Replenishment may be performed by any Fe—S assembly protein, such as SufE. SufE may transfer a sulfur atom from a cysteine to LipA. Other Fe—S cluster assembly proteins, such as IscR, IscS, IscU, IscA, HscB, HscA, IscX, IscI, IscII, Csd, SufA, SufB, SufC, SufD, SufE, SufS, NifS, NifU, and Nfs1 are also considered within the scope of the disclosures herein.

While not wishing to be bound by theory, it is possible that LipA and LipB may act in either order. LipB may act before LipA, or LipA may act before LipB. When LipA acts first, LipA may donate two sulfur atoms to octanoyl-ACP. Lipoic acid may be in a reduced form. Lipoic acid may be transformed into an oxidized form (wherein the two sulfurs are directly chemically bonded to each other) in an oxidizing environment.

A review on the biosynthesis of alpha lipoic acid in prokaryotes may be found in Chemistry & Biology, Vol. 11, January, 2004 and Miller et al., 2000, Biochemistry 39: 15166-15178.

### Nucleic Acids Useful in Producing Lipoic Acid

Genes involved in the production of lipoic acid are lipA, lipB, lplA, and Fe—S cluster assembly genes including sufE. The Gluconobacter oxydans 621H complete genome information is available in NCBl, under the project ID: 13325. R-alpha lipoic acid is synthesized in E. coli utilizing at least two important genes characterized as lipA and lipB. LipA is classified as lipoyl synthase and lipB is classified as lipoyl transferase. LipA protein is a catalytic enzyme and may have at least one Fe—S (iron—sulfur moiety) which is needed for its activity.

The Gluconobacter homologs of E. coli lipA, lipB, and sufE are known in the art. The Gene Accession ID of Gluconobacter lipoyl synthase (lipA) is 3249912 (SEQ ID No. 1), the Gene Accession ID of Gluconobacter lipoyl transferase (lipB) is 3248567 (SEQ ID No. 2), and the Gene Accession ID of Gluconobacter sufE is 3248646 (SEQ ID No. 3). All Gene Accession IDS refer to NCBI database numbers.

It will be readily apparent of one skilled in the art that one may make minor changes to the genes involved in lipoic acid synthesis without substantially altering the activity of the genes. Thus, mutations that do not substantially alter the activity of the genes involved in lipoic acid synthesis are contemplated within the scope of the methods described herein.

In one embodiment, a heterologous nucleic acid encoding a lipoic acid synthesis gene is used to replace all or a part of an endogenous lipoic acid synthesis gene. In another embodiment, the parent host cell is one that has been genetically modified to contain an exogenous lipoic acid synthesis gene; and the exogenous lipoic acid synthesis gene of the parent host cell is replaced by a modified lipoic acid synthesis gene that provides for a higher level of enzymatic activity, e.g., the amount of lipoic acid and/or the activity of the lipoic acid synthesis genes is higher than in the parent host cell.

Lipoic acid synthesis genes may be cloned using methods known in the art. For example, said genes may be amplified from genomic DNA or cDNA using forward and reverse primers by PCR. Methods of preparing genomic DNA and cDNA are known in the art. The amplified fragments may then be sequenced to verify that the fragment has the correct DNA sequence. DNA sequencing methods include die-terminator methods and variants thereof in conjunction with capillary electrophoresis.

As described below, one aspect of the systems and methods herein pertains to isolated nucleic acids comprising nucleotide sequences encoding lipoic acid synthesis polypeptides, for example as illustrated by SEQ ID No. 4-6, and/or equivalents of such nucleic acids. The term nucleic acid as used herein is intended to include fragments as equivalents. The term equivalent is understood to include nucleotide sequences encoding lipoic acid synthesis polypeptides which are functionally equivalent to the lipoic acid synthesis polypeptide encoded in SEQ ID Nos. 1-6. Equivalent nucleotide sequences will include sequences that differ by one or more nucleotide substitutions, additions or deletions, such as allelic variants; and will, therefore, include sequences that differ from the nucleotide sequence of the lipoic acid synthesis coding sequence of SEQ ID Nos. 1-6 due to the degeneracy of the genetic code. Equivalents will also include nucleotide sequences that hybridize under stringent conditions (i.e., equivalent to about 20-27° C. below the melting temperature (Tₘ) of the DNA duplex formed in about 1M salt) to the nucleotide sequences represented in SEQ ID Nos. 1-6.

Moreover, it will be generally appreciated that, under certain circumstances, it may be advantageous to provide homologs of a lipoic acid synthesis polypeptide which function in a limited capacity as one of either an agonist (e.g., mimics or potentiates a bioactivity of the wild-type lipoic acid synthesis protein) or an antagonist (e.g., inhibits a bioactivity of the wild-type lipoic acid synthesis protein), in order to promote or inhibit only a subset of the biological activities of the naturally-occurring form of the protein. Thus, specific biological effects can be elicited by treatment with a homolog of limited function.

Variants of the subject lipoic acid synthesis genes can be generated by mutagenesis, such as by discrete point mutation(s), or by truncation. For instance, mutation can give rise to variants which retain substantially the same, or merely a subset, of the biological activity of the lipoic acid synthesis polypeptide from which it was derived. Alternatively, antagonistic forms of the protein can be generated which are able to inhibit the function of the naturally occurring form of the protein. Thus, lipoic acid synthesis polypeptides provided herein may be either positive or negative regulators of an activity of an lipoic acid synthesis polypeptide.

In general, polypeptides referred to herein as having an activity of an lipoic acid synthesis polypeptide (e.g., are “bioactive”) are defined as polypeptides which include an amino acid sequence corresponding (e.g., at least 80%, 85%, 90%, 95%, 98%, 100% identical) to all or a portion of the amino acid sequences of the lipoic acid synthesis polypeptides described herein, such as tagged lipoic acid synthesis polypeptides, and which agonize or antagonize all or a portion of the biological/biochemical activities of a naturally occurring lipoic acid synthesis protein. Examples of such biological activity includes the ability to increase production of lipoic acid, the ability to regenerate the Fe—S cluster of a protein in need thereof, the ability to transfer an octanoyl group from one protein to another, and the ability to add sulfur moieties to an octanoyl group or a related nonphysiological substrate. The bioactivity of certain embodiments of the subject nucleic acids and polypeptides can be characterized in terms of an ability to increase production of lipoic acid and an ability to localize to an appropriate region of a cell.

Another aspect of the systems and methods herein provides a nucleic acid which hybridizes under high or low stringency conditions to a nucleic acid represented by one of SEQ ID Nos: 1-6. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

Nucleic acids having a sequence that differs from the nucleotide sequences shown in one of SEQ ID Ns. 4-6 due to degeneracy in the genetic code are also within the scope of the disclosures herein. Such nucleic acids encode functionally equivalent peptides but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence. However, it is expected that DNA sequence polymorphisms that do lead to changes in the amino acid sequences will also exist. One skilled in the art will appreciate that these variations in one or more nucleotides (up to about 3-5% of the nucleotides) of the nucleic acids encoding polypeptides having an activity of a lipoic acid synthesis polypeptide may exist among individuals of a given species due to natural allelic variation.

Fragments of the nucleic acids encoding an active portion of the lipoic acid synthesis proteins are also within the scope of the disclosure. As used herein, a lipoic acid synthesis gene fragment refers to a nucleic acid having fewer nucleotides than the nucleotide sequence encoding the entire amino acid sequence of an lipoic acid synthesis protein encoded by SEQ ID Nos. 1-6, yet which (preferably) encodes a peptide which retains some biological activity of the full length protein as described above. Nucleic acid fragments within the scope of the present application include those capable of hybridizing under high or low stringency conditions with the nucleic acids represented in SEQ ID Nos. 1-6. Nucleic acids within the scope of this disclosure may also contain linker sequences, modified restriction endonuclease sites and other sequences useful for molecular cloning, expression or purification of recombinant forms of the subject polypeptides.

### Vectors for Producing Lipoic Acid

In some embodiments, a heterologous nucleic acid is introduced into a parent host cell, and the heterologous nucleic acid recombines with an endogenous nucleic acid encoding a lipoic acid synthesis gene, thereby genetically modifying the parent host cell. In some embodiments, the heterologous nucleic acid comprises a promoter that has an increased promoter strength compared to the endogenous promoter that controls transcription of the endogenous lipoic acid synthesis genes, and the recombination event results in substitution of the endogenous promoter with the heterologous promoter. In other embodiments, the heterologous nucleic acid comprises a nucleotide sequence encoding a lipoic acid synthesis gene that exhibits increased enzymatic activity compared to the endogenous lipoic acid synthesis genes, and the recombination event results in substitution of the endogenous lipoic acid synthesis gene coding sequence with the heterologous lipoic acid synthesis gene coding sequence.

Cloned lipoic acid synthesis genes can be subcloned into a suitable vector. Such vector may be a plasmid exogenous to the host cell, a plasmid endogenous to the host cell, a cosmid, a bacterial artificial chromosome (BAC), a yeast artificial chromosome (YAC), a shuttle vector or any other appropriate vector. Preferably, the vector is permissive for high level expression and is stably maintained in the host cell. Smaller plasmids tend to be more stable than large ones, so a smaller plasmid is preferred. The plasmid can be selected on the basis of growth conditions and yield parameters. In certain embodiments, a shuttle vector with the cloned genes with optimized expression levels functional in both E.coli and Gluconobacter species can be constructed.

The plasmid may be a low, medium, or high copy number plasmid. Increasing the plasmid copy number is achieved by selecting a plasmid backbone that is known to be a medium or high copy number plasmid. Low copy number plasmids generally provide for fewer than about 20 plasmid copies per cell. Medium copy number plasmids generally provide for from about 20 plasmid copies per cell to about 50 plasmid copies per cell, or from about 20 plasmid copies per cell to about 80 plasmid copies per cell. High copy number plasmids generally provide for from about 80 plasmid copies per cell to about 200 plasmid copies per cell, or more.

In certain embodiments, lipoic acid synthesis genes are expressed as fusion proteins. A lipoic acid gene may be fused with (or operably linked to), for example, a tag directing its localization to a desired cellular compartment. In a preferred embodiment, lipoic acid synthesis genes are localized to the periphery of the host cell. This area may include the cell wall, the plasma membrane, the outer membrane and/or the periplasmic space. The localized genes may be membrane spanning, covalently linked to a molecule in the membrane, noncovalently bound to a molecule in the membrane, bound (covalently or noncovalently) to a component of the cell wall, or freely diffusible in the periplasmic space. The membrane referred to in this paragraph may be the cell membrane or the outer membrane.

In an especially preferred embodiment, a lipoic acid synthesis protein is tagged with a signal sequence that localizes it to the periplasmic space. The Gluconobacter oxydans genome contains a number of periplasmic enzyme-coding genes including dehydrogenases. Bio transformation reactions such as oxidation occur in the periplasm with the products being released in to the medium via membrane porins. A fusion protein comprising a membrane localization signal peptide along with at least one lipoic acid synthesis protein such as LipA may be expressed. Signal sequences may be cleaved during translocation of the exported protein across the lipid bilayer. Alternatively, one may fuse a membrane-spanning sequence to a lipoic acid synthesis protein in order to create a membrane-bound form of the protein.

For example, a periplasm-targeting sequence from one of the following proteins may be used as a tag: gene ID 476661 (SEQ ID No. 7) from plasmid ID GOX04504, gene ID 620902 (SEQ ID No. 8) from plasmid ID GOX0586, and gene ID 1072540 (SEQ ID No. 9) from plasmid ID GOX0973. The above-referenced genes are thought to be localized to the periplasmic space, and the localization-directing portions are usually found at the 5′ or 3′ end. Therefore, an amino acid sequence of between about 15 and 30 amino acids from the N- or C-terminal portions of these proteins may be used to target lipoic acid synthesis genes to the periplasmic space. The tag sequence may be fused to the 5′ or 3′ end of the lipoic acid synthesis gene. In a preferred embodiment, a tag derived from the N-terminus of an endogenous protein is fused to the N-terminus of the lipoic acid synthesis gene and a tag derived from the C-terminus of an endogenous protein is fused to the C-terminus of the lipoic acid synthesis gene.

Genes for lipoic acid synthesis may be added to any appropriate vector. Any appropriate vector should be permissive for maintenance in Gluconobacter. In a preferred embodiment, the vector can also be maintained in E. coli. It may be useful to perform subcloning steps in E. coli. Examples of suitable plasmids include pGE1 and _{P}GE2. These are shuttle vectors that can replicate both in G. oxydans and E. coli. Another suitable vector is pGO32935 which is an endogenous Gluconobacter plasmid. Other suitable plasmids include pSUP301, pVK102, pGOX1, pGOX2, pGPX3, pGOX4, and pGOX5.

One may control expression of lipoic acid synthesis genes using any appropriate promoter. An appropriate promoter may be inducible, constitutive, or repressible. Acceptable promoters include rRNAB PI, P (from bacteriophage lambda), Pₐₙₜ (from bacteriophage P22), P_{spc}, P_{bla}, P1, P2, T3, T7, tufB, any Gluconobacter ribosomal gene promoter, pTrp, pTac, PL, PT7, pBAD, and pLac. It will be apparent to one of skill in the art that certain mutations may be made to said promoters without substantially affecting their function. Promoters with such mutations are encompassed within the scope of the methods herein.

One may choose an appropriate promoter to provide optimal production of lipoic acid. For example, if high levels of lipoic acid production do not impair cell growth or viability, one may choose a constitutive promoter to drive expression of lipoic acid synthesis genes. If lipoic acid does impair cell growth or viability, or has any other unwanted effect, one may choose an inducible or repressible promoter. Thusly, one may prevent high-level production of lipoic acid when the culture is growing. When lipoic acid production is desired, one may add an agent that induces gene expression to the medium to stimulate expression from an inducible promoter. Alternatively, one may remove an agent that represses gene expression from the medium to allow expression from a repressible promoter.

Vectors may comprise one or more of a transcription initiation or transcriptional control region(s) (e.g., a promoter), the coding region for the protein of interest, and a transcriptional termination region. Transcriptional control regions include those that provide for over-expression of the protein of interest in the genetically modified host cell; those that provide for inducible expression, such that when an inducing agent is added to the culture medium, transcription of the coding region of the protein of interest is induced or increased to a higher level than prior to induction. Transcriptional control regions also include those that provide for repression of the protein of interest.

If lipoic acid is produced in yeast, a number of vectors containing constitutive or inducible promoters may be used. For a review see, Current Protocols in Molecular Biology, Vol. 2, 1988, Ed. Ausubel, et al., Greene Publish. Assoc. & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516 544; Glover, 1986, DNA Cloning, Vol. 11, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673 684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (Cloning in Yeast, Ch. 3, R. Rothstein In: DNA Cloning Vol. 11, A Practical Approach, Ed. D M Glover, 1986, IRL Press, Wash., D.C.). Alternatively, vectors may be used which promote integration of foreign DNA sequences into the yeast chromosome.

Other regulatory elements such as enhancers of transcription and favored translation initiation sequences may also be used in keeping with the methods herein.

In a preferred embodiment, the vector contains at least one selectable marker. A selectable marker may be used, for example, to isolate a strain of transgene-bearing bacteria from a heterologous population, such as a population generated by a transformation. This marker may be an antibiotic-resistance gene or a gene that complements an auxotrophic mutation. Two suitable selectable markers are ampR (ampicillin-resistance) and genR (gentamicin resistance). Other acceptable markers are emR (erythromycin resistance), kanR (kanamycin resistance), chlorR (chloramphenicol resistance), smR (streptomycin resistance), tetR (tetracycline resistance), leu+ (complements leucine auxotrophy), ura+ (complements uracil auxotrophy), trp+ (complements tryptophan auxotrophy), his+ (complements histidine auxotrophy), lys+ (complements lysine auxotrophy), met+ (complements methionine auxotrophy), and ade+ (complements adenine auxotrophy). Selection may also use expression of a marker such as GFP, luciferase, or beta-galactosidase that has a visual phenotype rather than a cell survival phenotype. The selectable marker may be under the control of any promoter listed above. Preferably, the promoter is a constitutive promoter.

The vector may contain a suitable origin of replication. A suitable origin may function in Gluconobacter, in E. coil, or both. An origin of replication may be selected from the group consisting of OriV and the origins found on plasmids pACYC184, RP4, RSF1010, pBR322, pACYC177, pACYC184, pSC101, pGE1, pGE2, pGO32935, pSUP301, pVK102, pMB1, and the origins found on bacteriophages lambda, P 1, and T-coliphages. Also, any origin from the main Gluconobacter chromosome or endogenous plasmids may be used. Broadly, any origin that functions in the host strain may be used.

The vector may include any other sequences that support plasmid stability and/or gene expression. Such sequence include polyadenylation sequences, transcriptional terminators, sequences that promote integration into the host genome, and sequences that promote equal segregation of plasmids during cell division.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

This application also provides expression vectors (also called vectors or constructs) containing a nucleic acid encoding a lipoic acid synthesis polypeptide, operably linked to at least one transcriptional regulatory sequence. Operably linked is intended to mean, in this context, that the nucleotide sequence is linked to a regulatory sequence in a manner which allows expression of the nucleotide sequence. Regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences, sequences that control the expression of a DNA sequence when operatively linked to it, may be used in these vectors to express DNA sequences encoding the polypeptides disclosed herein. Such useful expression control sequences, include, for example, a viral LTR, such as the LTR of the Moloney murine leukemia virus, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs of the present disclosure can also be used to deliver nucleic acids encoding the subject polypeptides. Thus, another aspect of the described systems and methods features expression vectors for in vivo or in vitro transfection and expression of a subject polypeptide in particular cell types.

Expression constructs of the subject polypeptide, including agonistic and antagonist variants thereof, may be administered in any biologically effective carrier, e.g. any formulation or composition capable of effectively delivering the recombinant gene to cells in vivo or in vitro. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation. One of skill in the art can readily select from amongst available vectors and methods of delivery in order to optimize expression in a particular cell type or under particular conditions.

### Microorganisms Suitable for Producing Lipoic Acid

The nucleic acids and proteins described herein may be expressed in any appropriate host cell. The methods herein may be practiced using any acceptable host cell. Acceptable host cells include acid-tolerant microorganisms such as certain bacteria and yeast.

A preferred embodiment envisions the use of bacterium of the genus Gluconobacter to for producing lipoic acid. Gluconobacter displays strong acid tolerance. Since production of large amounts of lipoic acid will strongly acidify the culture medium, an acid-tolerant bacterium is expected to withstand higher levels of lipoic acid than a non-acid tolerant bacterium. In addition, culture medium may be acidic if it comprises octanoic acid or any acidic precursor. An especially preferred embodiment will use Gluconobacter oxydans.

Certain embodiments encompass the use of the following bacteria, with the strain identification number in parentheses: Gluconobacter oxydans (IFO3189, IFO12467), Gluconobacter suboxydans (IFO3254, IFO3255, IFO3256, IFO3257, IFO12528), Gluconobacter melanogenus (IFO3292, IFO3293, IFO3294), Gluconobacter albidus (IFO3250, IFO3253), Gluconobacter capsulatus (IFO3462), Gluconobacter cerinus (IFO3263, IFO3264, IFO3265), Gluconobacter dioxyacetonicus (IFO3271, IFO3274), Gluconobacter gluconicus (IFO3285, IFO3286), Gluconobacter industrius (IFO3260), and Gluconobacter nonoxygluconicus (IFO3275, IFO3276). Other microorganisms that may be used include species of the Lactobacillus genus, species of the Acetobacter genus (including Acetobacter aceti), species of the Azotobacter genus, species of the Bacillus genus (including Bacillus megaterium), species of the Ervinia genus (including Ervinia carotovora), and species of the Thiobacillus genus (including Thiobacillus ferrooxidans).

Other appropriate microorganisms for producing lipoic acid using the methods herein include Enterobacteriae including E. coli, and yeasts including yeasts of the genus Saccharomyces (such as S. cerevisiae), the genus Schizosaccharomyces (such as S. pombe), and Pichia (such as P. pastoris). Suitable eukaryotic host cells for producing lipoic acid include, but are not limited to, yeast cells, insect cells, plant cells, fungal cells, and algal cells. Suitable eukaryotic host cells include, but are not limited to, Pichia pastoris, Pichiafinlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kltryveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some embodiments, the host cell is a eukaryotic cell other than a plant cell.

Applicants herein disclose methods of transgenically expressing combinations of lipoic acid synthesis genes in acid-tolerant microorganisms. One skilled in the art may further assay lipoic acid production of such transgenes using methods known in the art. In one embodiment, one may express exactly one of lipA, lipB, and a Fe—S cluster assembly gene. In a preferred embodiment, the Fe—S cluster gene will be sufE. In an alternative embodiment, one may express two of lipA, lipB, and a Fe—S cluster assembly gene. In yet another embodiment, one may express all three of lipA, lipB, and a Fe—S cluster assembly gene. In another embodiment, one may express any of the above combinations of genes together with lplA. Based on the disclosures herein, one of skill in the art may readily design a plasmid containing the minimum number of genes necessary to produce sufficiently high levels of lipoic acid.

One may select the optimal combination of lipoic acid synthesis genes using the methods described herein. For example, if expression of LipA alone results in insufficient production of lipoic acid, an Fe—S cluster assembly gene may be added to the genetically modified strain. An Fe—S cluster assembly gene may boost the activity of LipA. It may do so by increasing the proportion of LipA that is in the active Fe₄S₄ form.

Enzymatic activity of lipoic acid synthesis genes may be assayed using methods known in the art. For example, the activity of LipA may be assayed as described in Bryant et al., “The activity of a thermostable lipoyl synthase from Sulfolobus solfataricus with a synthetic octanoyl substrate” Anal Biochem. 2006 Apr 1;351(1):44-9. Epub 2006 Feb. 3. In this method, one uses an in vitro assay for LipA activity using a synthetic tetrapeptide substrate, containing an N(epsilon)-octanoyl lysine residue, corresponding in sequence to the lipoyl binding domain of the E2 subunit of pyruvate dehydrogenase. The activity of LipB may be assayed in vitro by determining its ability to transfer an octanoyl group from octanoyl-ACP to apo-H protein as described in Nesbitt NM et al, “Expression, purification, and physical characterization of Escherichia coli lipoyl(octanoyl)transferase. ” Protein Expr Purif. 2005 February; 39(2):269-82. The activity of SufE or other Fe—S cluster assembly proteins may be determined by their ability to promote LipA activity in a purified system, wherein LipA activity is assayed using the method of Bryant et al. above. An additional measure of SufE functionality is its ability to bind SufB as described in Layer G et al., “SufE transfers sulfur from SufS to SufB for iron-sulfur cluster assembly. “J Biol Chem. 2007 May 4; 282(18):13342-50. Epub 2007 Mar 9.” The activity of LplA may be measured using an assay in which purified LplA is allowed to catalyze the ATP-dependent attachment of [35S]lipoic acid to apoprotein (Morris TW et al, “Identification of the gene encoding lipoate-protein ligase A of Escherichia coli. Molecular cloning and characterization of the lplA gene and gene product. ” J Biol Chem. 1994 Jun. 10; 269(23):16091-100.)

In certain embodiments, lipoic acid synthesis genes are integrated into the genome of the microorganism. This integration may be performed using homologous recombination, site-specific recombination, transposition, or any other method known in the art. The integrated genes may be present in one copy or multiple copies.

The vector bearing lipoic acid synthesis genes may be introduced into a host cell using any method known in the art. For example, a shuttle vector may be used for efficient transformation. A vector also may be introduced into a host cell by conjugation or by using techniques such as electroporation or chemical-mediated transformation such as CaCl₂ mediated transformation. Transformation may involve a heat-shock protocol.

Selection of transformants may be performed based on any method known in the art. Selection may be performed, for example, using antibiotic selection for a marker gene, selection for prototrophy of a given marker (i.e. a marker that complements an auxotrophic phenotype), or via PCR using plasmid or vector probes to detect the DNA of interest. Other methods to identify transformants include Southern blots, Northern blots, and ribopattern analysis. Selection may also be performed by assaying the ability of the bacteria to produce lipoic acid.

In a preferred embodiment of isolating transformed cells, genetically engineered G. oxydans may be cultivated in MB medium with antibiotics in a shaker flask at 30° C. for 24 hrs. Two percent of the broth is then transferred to fresh medium without antibiotics and the culture is grown for 2 days at 30° C. Samples of G. oxydans grown in antibiotic-free broth are plated on antibiotic-free MB-agar medium, incubated at 27° C. for 5 days. Finally, the grown colonies are streaked on to antibiotic MB-agar medium, and thus colonies are selected. Resulting colonies can also be analyzed using any method known in the art, including Southern blots, Northern blots, or ribopattern analysis.

Constructs for expression or overexpression of proteins as described above may be introduced into the host cell by any methods known in the art. Any means for the introduction of polynucleotides into eukaryotic or prokaryotic cells may be used in accordance with the compositions and methods described herein. Suitable methods include, for example, direct needle microinjection, transfection, electroporation, retroviruses, adenoviruses, adeno-associated viruses; Herpes viruses, and other viral packaging and delivery systems, polyamidoamine dendrimers, liposomes, and more recently techniques using DNA-coated microprojectiles delivered with a gene gun (called a biolistics device), or narrow-beam lasers (laser-poration). In one embodiment, nucleic acid constructs may be delivered in a complex with a colloidal dispersion system. A colloidal system includes macromolecule complexes, nanocapsules, microspheres, beads, and lipid-based systems including oil-in-water emulsions, micelles, mixed micelles, and liposomes. An exemplary colloidal system is a lipid-complexed or liposome-formulated DNA. See, e.g., Canonico et al., Am J Respir Cell Mol Biol 10:24-29, 1994; Tsan et al., Am J Physiol 268 (6 Pt 1): 1052-6 (1995); Alton et al., Nat Genet. 5:135-142, 1993 and U.S. Pat. No. 5,679,647 by Carson et al.

### Microorganisms Optimized for Lipoic Acid Production

A genetically modified host cell suitable for use in a subject method is genetically modified with one or more nucleic acids, including a nucleic acid comprising a nucleotide sequence comprising a lipoic acid synthesis gene, such that the level of that gene's expression is increased. The level of that gene's expression is increased by at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, compared to a wild-type cell of the same species cultured under comparable conditions. Furthermore, the level of the gene's expression may be 1.5, 2, 5, 10, 20, 50, 100, 200, 500, 1000, 2000, 5000, or 10000 or more-fold greater than that of a wild-type cell of the same species cultured under comparable conditions. Levels of gene expression may be assayed using techniques known in the art such as, for example, Northern blotting, quantitative RT-PCR, and Western blotting.

In some embodiments, microorganisms producing lipoic acid may be optimized for desirable characteristsics. Such characteristics include a rapid growth rate, a high rate and/or level of production of lipoic acid, and strong tolerance to acidic conditions. Growth of genetically modified host cells is readily determined using well-known methods, e.g., optical density (OD) measurement at about 600 nm (OD₆₀₀) of liquid cultures of bacteria; colony size; growth rate; and the like.

One method of optimizing lipoic acid production is by gene codon optimization. Examples of codon-optimized genes are shown in SEQ ID Nos. 4, 5, 6 and are discussed in Example 1. The codon-optimized sequences have significant sequence differences from the corresponding wild-type sequences. In some embodiments, the nucleotide sequence encoding a lipoic acid synthesis enzyme is modified such that the nucleotide sequence reflects the codon preference for the particular host cell. For example, the nucleotide sequence will in some embodiments be modified for yeast codon preference. See, e.g., Bennetzen and Hall (1982) J. Biol. Chem. 257(6): 3026 3031. The nucleotide sequence may be codon-optimized for expression in any acid-tolerant microorganism.

Based on the disclosures herein and the state of the art, one of skill in the art would readily be able to determine the codon-optimized sequence of other genes such as lplA and Fe—S cluster assembly genes. Based on the disclosures herein and the state of the art, one of skill in the art would readily be able to determine the codon-optimized sequence lipoic acid synthesis genes in other acid-tolerant microorganisms as well.

Once a codon-optimized sequence has been determined, the corresponding physical DNA molecule may be produced using methods known in the art. For example, PCR amplification using mutant primers can be used to generate the desired mutations in the wild-type gene. Additionally, genes may be synthesized in vitro using, for example, the methods disclosed in U.S. Patent Application Nos. U.S. 2007-0004041 A1, U.S. 2006-0194214 A1, and U.S. 2006-0035218 A1.

Multiple gene sequences with differing codon usage may be tested. One may assay transcript levels, protein levels, enzymatic activity, or lipoic acid levels using methods known in the art. Based on these assays, one may experimentally determine the DNA sequence that results in the highest lipoic acid production and/or lowest toxicity to the host cell, or other desirable characteristics.

Strains may also be optimized for lipoic acid production by mutant strain selection. One may express lipoic acid synthesis genes in a variety of mutant organisms, and assay for lipoic acid production.

Strains may also be optimized for lipoic acid production by artificial selection. For instance, one may select for a strain that displays a faster growth rate or better tolerance to acidic conditions. One may grow the culture for multiple generations in medium containing lipoic acid, in order to select for improved mutants. Strains may be selected for improved hardiness including resistance to acidic conditions, resistance to high temperatures, or for an increased rate of cell division, according to known methods.

Selection of industrially potent strains may be done via an individual colony screening process by determining the expression rate and production rate of R-alpha-lipoic acid. Multiple transformed cells expressing (including overexpressing) lipoic acid synthesis genes may be grown in separate culture vessels. If necessary, expression of the appropriate genes will be induced. Then, lipoic acid levels may be measured. Alternatively, one may measure expression of lipoic acid synthesis genes. In this manner one may determine which strains produce the highest levels of lipoic acid.

Lipoic acid levels may be determined using any method known in the art. Production of R-(+)-alpha lipoic acid is determined using TLC, HPLC or gas chromatography. In addition, lipoic acid levels may be determined using the known turbidometric bioassay (Herbert and Guest, 1970, Meth. Enzymol., 18A, 269-272).

One may further optimize a strain for lipoic acid production by selecting a vector such as a plasmid with high stability. Multiple plasmids may be tested; examples of suitable plasmids are listed in the instant application. Plasmid stability may be determined by growing the host cells in the non-selective medium, and then plating the cells on two plates, one with selective conditions and one without. Comparing the number of colonies on the two plates will indicate what proportion of cells lost the selectable marker and hence the plasmid of interest.

Microorganisms may be optimized using any methods known in the art, including those described in “Functional Genetics of Industrial Yeasts” by Johannes H. de. Winde, Springer (Aug. 13, 2003).

### Methods of Culturing Microorganisms

Depending on the vector used and the growth conditions and medium for growth suitable strains of Gluconobacter species or genus may be selected. Physical and chemical parameters may be optimized. The amount of time cultures are kept shaker flasks may be determined depending on the experimental parameters.

One may adapt culture conditions for optimal lipoic acid production in several ways. One may optionally add co-factors, co-enzymes, or minerals to the medium. For example, at least one of ATP, SAM, NADPH, and flavodoxin may be added to the medium. The base components of the medium may also be altered. A preferred culture medium is MB. MB comprises (per liter): 25 g mannitol, 5.0 g yeast extract, and 3.0 g Bactopeptone. Appropriate culture conditions are also disclosed in Wei S, Song Q, and Wei D. ‘Production of Gluconobacter oxydans cells from low-cost culture medium for conversion of glycerol to dihydroxyacetone’. Prep Biochem Biotechnol, 2007; 37(2):113-21. Culture conditions may differ from the specifics listed above without departing from the scope of the methods herein.

In addition, the temperature of the culture conditions may be optimized. Preferred embodiments will set the temperature of a Gluconobacter culture at between 25° C. and 30° C., although other temperatures may be used according to the present methods. The pH of the medium may also be altered. A preferred pH of culture medium is 7.2. A preferential initial range is between 6 and 8, or between 5 and 9. It is expected that once high-level lipoic acid synthesis begins, the pH of the medium will drop below to approximately pH 3 or 4, but may also be in the range of pH 2 to 5, or pH 2 to 6. Furthermore, addition of octanoic acid to the medium may also cause the pH of the medium to drop below approximately pH 3 or 4, but may also be in the range of pH 2 to 5, or pH 2 to 6. Values for temperature and pH may also be outside the ranges listed above without departing from the scope of the methods herein.

Lipoic acid-producing cells may be grown using any mechanical culture apparatus known in the art. An appropriate apparatus may be, for example, a rolling shaker or an orbital shaker (for example, adapted for holding shaker flasks). The rotation speed of a shaker may be 150-400 rpm, 100-500 rpm, 150-250 rpm, 150-200 rpm, or about 170 rpm. Values for shaker speed may also be outside the ranges listed above without departing from the scope of the methods herein.

If necessary, appropriate inducers and/or repressors may be added to the culture medium. If lipoic acid synthesis genes are controlled by an inducible promoter, an inducer may be added to the medium when lipoic acid synthesis is desired. If lipoic acid synthesis genes are controlled by a repressible promoter, a repressor may be withdrawn when lipoic acid synthesis is desired. For instance, IPTG may be added to induce gene expression driven by pLac, pTrc, pTac, or PT7. Arabinose may be added to induce expression driven by pBAD. IAA may be added to induce expression driven by pTrp. Nalidixic acid may be added to induce expression driven by PL (also, heat may be applied to induce expression from PL). Octanoic acid may be added to induce expression driven by endogenous promoters of lipoic acid synthesis genes. Tryptophan may be added to repress expression driven by pTrp.

Various lipoic acid precursors may be added to the culture medium. A preferred precursor is octanoic acid. Other precursors include octanoic acid, octanoate, or an octanoylated molucule such as octanoyl-AMP. Yet other precursors include intermediates of the fatty acid synthesis pathway. One may also add to the medium any combination of octanoic esters or caprylic aldehyde or alcohol or any substrate or any metabolite or any chemical solvent or any carbohydrate. Such molecules may be provided as precursors of lipoic acid and/or nutrient sources for the cultured microorganism.

Various antibiotics may be added to the culture medium. Wild-type Gluconobacter are sensitive to streptomycin. If a desired gene is marked with a streptomycin resistance marker, streptomycin may be added to the medium to prevent loss of the transgene. Similarly, ampicillin or an analog thereof may be added to the medium to prevent loss of an ampR-linked transgene. Other antibiotics that may be used include erythromycin, kanamycin, chloramphenicol, and tetracycline, penicillin, and analogs thereof. Antibiotics may be used, for example, to prevent loss of a transgene and/or plasmid. Antibiotics may also be used to prevent the growth of unwanted organisms in the culture.

Growth medium composition may be changed depending on the target molecule R-(+)-alpha lipoic acid production. Ingredients may be added or removed for optimization of production.

In some embodiments, the amount of lipoic acid produced by the genetically modified host cells described herein is greater than the amount of lipoic acid produced by corresponding wild-type cells cultured under comparable conditions. For example, the genetically modified cells may produce 1.5, 2, 5, 10, 20, 50, 100, 200, 500, 1000, 2000, 5000, or 10000 or more-fold more lipoic acid than the corresponding wild-type cells. In addition, the genetically modified cells may produce at least 100 pg, 200 pg, 500 pg, 100 ng, 200 ng, 500 ng, 100 ug, 200 ug, 500 ug, 1 mg, 2 mg, 5 mg, 10 mg, 20 mg, 50 mg, 100 mg, 200 mg, 500 mg, or 1 g or more of lipoic acid per gram dry cell weight of the engineered organism. In some embodiments, the concentration of lipoic acid in the culture medium is greater than 0.1, 0.2, 0.5, 0.75, 1, 2, 5, 20, 50, or 100 mg/ml.

Production of Valuable Secondary Metabolites from the Spent Medium

After the extraction of lipoic acid, many valuable secondary metabolites may remain in the culture medium. The medium may include nutrients, essential cofactors, and coenzymes. This medium can be scaled up for the generation of valuable metabolites such as, for example, acids, aldehydes, gluconates, and sugars in various isomerized forms. Specific secondary metabolites include L—sorbose (produced from D—sorbose and used as a precursor for Vitamin C synthesis), D—gluconic acid, 5—keto- and 2—ketogluconic acids (made from D—glucose and used as a precursor for various commercial solvents production), Dihydroxyacetone (produced from acetone and used in cosmetics as a sunless tanning product), and D-fructose (made from D-sorbitol and used as a sweetener). Secondary metabolites may be isolated using methods known in the art.

Gluconobacter species have oxidizing and reducing enzymes (oxidoreductases) on the periplasmic membrane. Thus, certain metabolic reactions occur in the periplasmic space and the metabolites are released into the medium. This reduces the risk of cytolysis, which is a source of major contaminant for target metabolite purification. Thus, bacteria that express lipoic acid synthesis genes in the periplasmic space are expected to produce purer lipoic acid than bacteria that express these enzymes intracellularly.

### Utility of the Disclosed Systems and Methods

The methods described herein of producing lipoic acid have broad utility. For example, one may make bulk quantities of lipoic acid for use in a pharmaceutical preparation, nutritional supplement, or neutraceutical. One may also manufacture lipoic acid for the purpose of studying lipoic acid or its effects on cells or organisms.

Alpha lipoic acid may be used for the alleviation of symptoms as well as the treatment of many diseases like diabetes, glaucoma, neuropathy etc. because of its antioxidant and free radical scavenging abilities. For a review refer: Free Radical Biology R Medicine. Vol. 19, No. 2, 227-250, 1995 and Toxicology and Applied Pharmacology 182, 84-90, 2002.

The practice of the present methods will employ, unless otherwise indicated, conventional techniques of cell biology, cell culture, molecular biology, transgenic biology, microbiology, recombinant DNA, and immunology, engineering, robotics, optics, computer software and integration. The techniques and procedures are generally performed according to conventional methods in the art and various general references. which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Patent No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986); Lakowicz, J. R. Principles of Fluorescence Spectroscopy, New York:Plenum Press (1983), and Lakowicz, J. R. Emerging Applications of Fluorescence Spectroscopy to Cellular Imaging: Lifetime Imaging, Metal-ligand Probes, Multi-photon Excitation and Light Quenching, Scanning Microsc. Suppl VOL. 10 (1996) pages 213-24, for fluorescent techniques, Optics Guide 5 Melles Griot® Irvine Calif. for general optical methods, Optical Waveguide Theory, Snyder & Love, published by Chapman & Hall, and Fiber Optics Devices and Systems by Peter Cheo, published by Prentice-Hall for fiber optic theory and materials.

### Exemplification

### Example 1

### Design of Codon-Optimized Lipoic Acid Synthesis Genes.

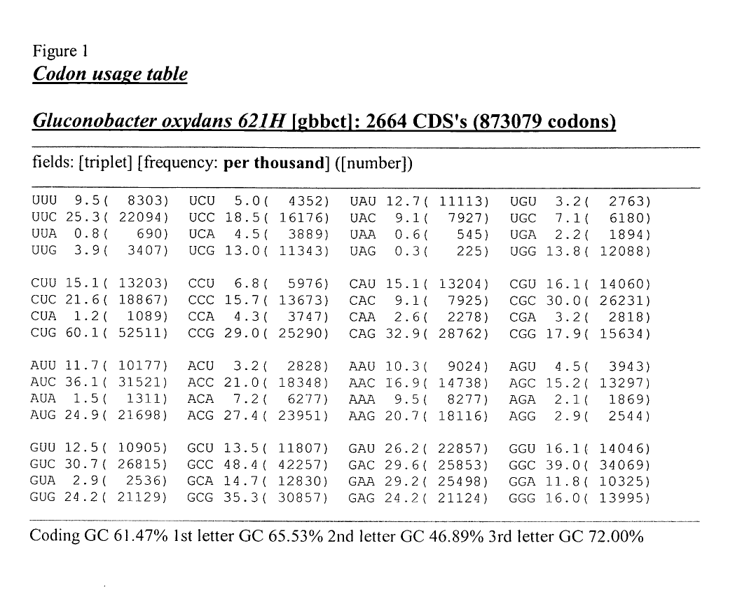

Lipoic acid synthesis genes, optimized for expression in in Gluconobacter oxydans, were designed and synthesized. The wild-type sequence of LipA is shown in SEQ ID No. 1, the wild-type sequence of LipB is shown in SEQ ID No. 2, and the wild-type sequence of SufE is shown in SEQ ID No. 2. The wild-type nucleotide sequences for LipA, LipB and SufE genes were taken from the genome sequence of G. oxydans (Genbank# NC_{—}006677). The LipA (Lipoyl synthase) gene is encoded in the region of 2513988bp-2514956bp (969 bp) in G. oxydans's genome. LipB (Lipoyltransferase) is encoded in the region 51862bp-52563bp (702 bp) and SufE gene is encoded in region 283898-284332 (435bₚ).The wild-type sequences were optimized using a table of the codon frequency usage in G. oxydans, depicted in FIG. 1. The resulting codon-optimized sequence for LipA is listed as SEQ ID No. 4. The resulting codon-optimized sequence for LipB is listed as SEQ ID No. 5. The resulting codon-optimized sequence for SufE is listed as SEQ ID No. 6. The codon-optimized sequences have significant sequence differences from the corresponding wild-type sequences.

### Example 2

### Construction of Vectors Expressing Lipoic Acid Synthesis Genes

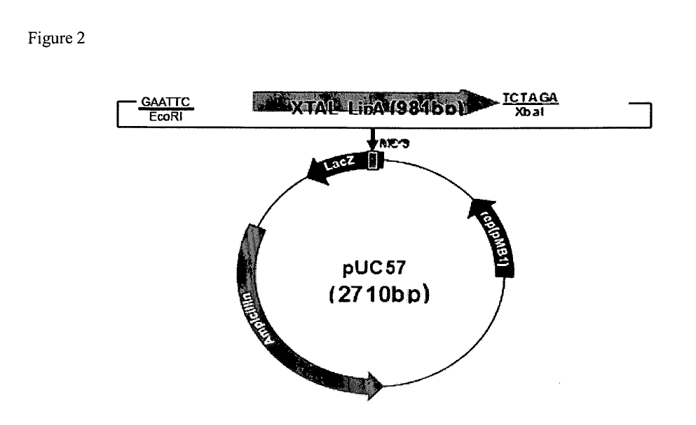

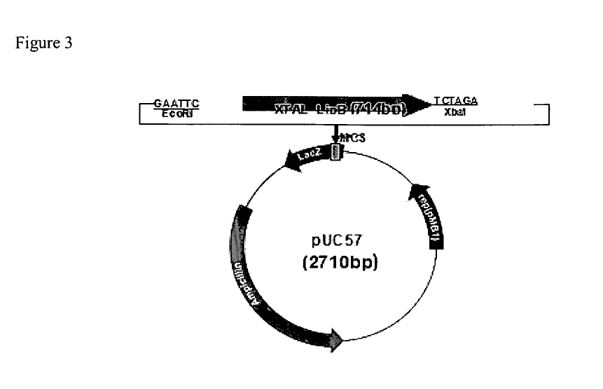

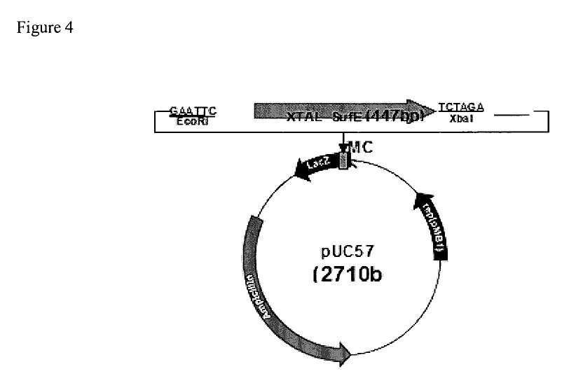

The codon-optimized genes were synthesized and cloned into pUC57 E. coli vectors, using EcoRI and XbaI sites for sequence analyses. These vectors have an ampicillin resistance marker. The promoter pTac was used to drive expression of the genes. The maps of these plasmids are shown in FIGS. 2, 3, and 4.

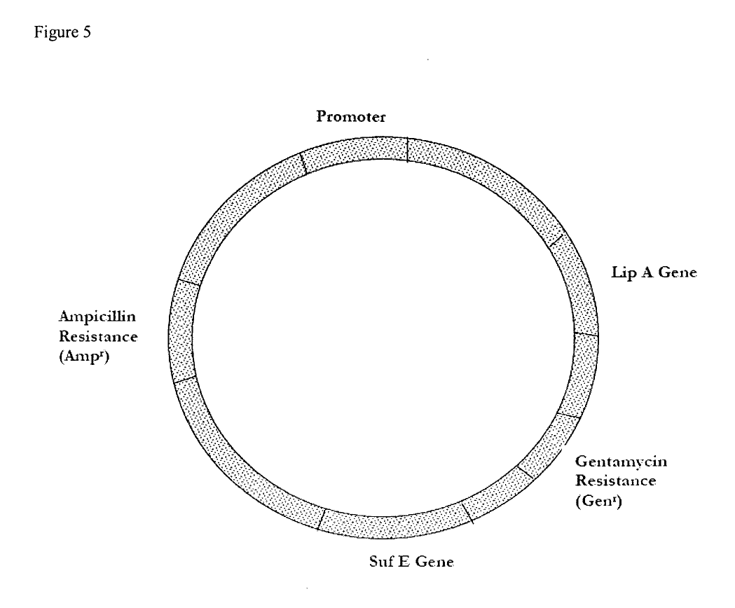

Next, a G. oxydans expression vector with both LipA and SufE was produced. The pUC57 vector expressing LipA was digested, and nucleic acids encoding SufE and gentamicin resistance were inserted. SufE and LipA expression may be driven with a pTac or tufB promoter. This vector is diagrammed in FIG. 5.

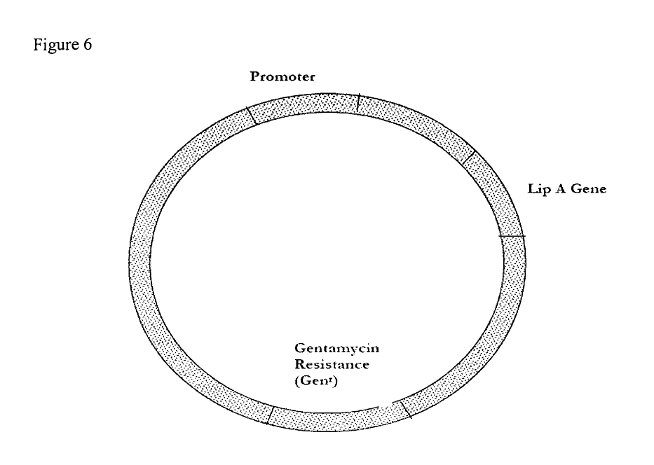

Next, a G. oxydans expression vector with LipA and a gentamicin resistance marker was produced. SufE and LipA expression may be driven with a pTac or tufB promoter. This vector is diagrammed in FIG. 6.

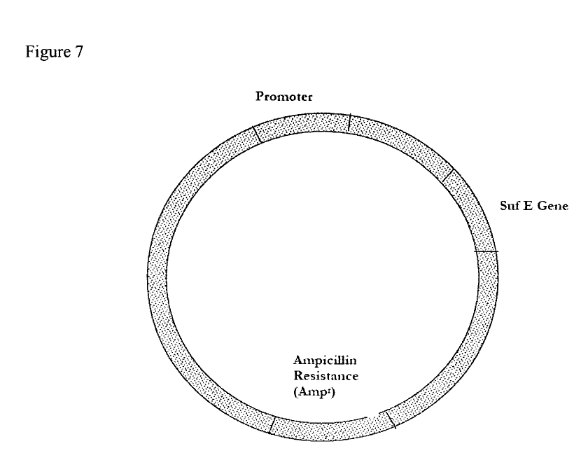

Finally, a G. oxydans expression vector with SufE and an ampicillin resistance marker was produced. SufE expression may be driven with a pTac or tufB promoter. This vector is diagrammed in FIG. 7.

### Example 3

### Transformation of Vectors into Gluconobacter oxydans

A Gluconobacter oxydans strain was purchased from ATCC (621H) and cultures were grown in mannitol media (25 g/L D(+) mannitol, 5 g/L yeast extract, 3 g/L peptone and pH adjusted to 6.0 with HCl). Cultures were grown in shaker flasks at 170 rpm at 26° C.

Chemically competent Gluconobacter oxydans cells were made for transformations with the plasmids. Cells were grown to OD₆₀₀ of 0.4 and harvested. The resulting cell pellet was washed with 0.1 M MgCl₂, resuspended in 0.1 M CaCl₂ and incubated in ice for 1 hour to establish competency. Cells were dispensed into 100 μL aliquots, flash frozen and stored at −80° C.

Plasmids were transformed into chemically competent Gluconobacter oxydans by the heat shock method and plated on mannitol-agar plate containing appropriate antibiotics (7 μg/mL of Gentamicin or 50 μg/mL of ampicillin). In one experiment, the plasmid encoding LipA was transformed. In addition, the plasmid encoding LipA and SufE was transformed. Plates were incubated at 30° C. for 3-4 days, and single colonies resulted.

After LipA transformation, a single colony of Gluconobacter oxydans containing LipA/pEXGOX-G was picked and grown in mannitol media containing gentamycin to OD₆₀₀ of 0.4-0.6. Chemically competent cells were made by the same protocol as described above. The SufE/pEXGOX-A plasmid described above was transformed into this competent cell to obtain double transformed Gluconobacter oxydans.

### Example 4

### Analysis of Lipoic Acid

Two different G. oxydans strains expressing LipA and SufE were cultured, and the resulting culture broth was analyzed by HPLC/MS. The first strain, described in Example 2, contained lipA and sufE in the same plasmid. The second strain, also described in Example 2, contained lipA and sufE in separate plasmids. Pure lipoic acid was used as a standard. Four different broths, obtained by culturing the bacteria for different lengths of time, were analyzed by HPLC/MS. All HPLC were run in APCI negative mode with a gradient eluent of 95% water/5% acetonitrile to 5% water/95% acetonitrile (+0.1% formic acid).

For the lipoic acid standard, a single UV-active peak was seen at 11.05 - 12.5 minutes. This peak corresponds to a MW=of 203.03 to 205. MS of the 11.05 or 12.5 minute peak showed MW=203.03 to 205 (M−1 of lipoic acid) present as the largest mass peak.

In order to estimate the lower limit for detection of lipoic acid in our HPLC system, two experiments were run with the culture broths with known amount of lipoic acid as internal standard of 1 mg/mL and 0.1 mg/mL. At 1.0 mg/mL (HPLC/MS) the lipoic acid could clearly be seen and at 0.1 mg/mL (HPLC/MS), the lipoic acid could still be seen.

The amount of lipoic acid in the culture broths described above was quantified as approximately 1.0 mg/ml with 11.05 min retention time in HPLC and m/e (negative) 205.03 in mass spectroscopy.

### Example 5

### Cloning into a Dual Vector System

In order to measure the expression levels of lipoic acid in a different host cell, E. coli, LipA and SufE genes were PCR amplified, and cloned into an E. coli dual vector system, pCDF-Duet1. The LipA gene was cloned into MCS1 using EcoRI and Sall sites. Separately, SufE gene was cloned into MCS2 using NdeI and XhoI sites. After verifying the nucleotide sequences, the SufE gene was cut and re-ligated into pCDF-Duet1 vector containing LipA gene to yield the dual expression plasmid. Such dual use plasmids may be used, for instance, for measuring the levels of LipA and SufE in E. coli.

### Equivalents

The present disclosure provides among other things compositions and methods for metabolic engineering. While specific embodiments of the subject disclosure have been discussed, the above specification is illustrative and not restrictive. Many variations of the systems and methods herein will become apparent to those skilled in the art upon review of this specification. The full scope of the claimed systems and methods should be determined by reference to the claims, along with their full scope of equivalents, and the specification, along with such variations.

### Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

SEQUENCE LISTING

SEQ ID No. 1: LipA
Gene Accession ID: 3249912 (lip A) lipoyl synthase. (Source: NCBI)
Gluconobacter oxydans 621H, complete genome.
Coding nucleic acid sequence:
ATGTCCCAGCGCATCACCATCGATCACCGTTCGGCGCCTGCCCTTCGCCATCCGGAGAAGGC
GCATCGGCCTGATAACCCGATCCAGCGCAAGCCGTCCTGGATCCGGGTCAAGGCGCCGAACC
ATCCAGTCTATCACGAGACCCGTGCGCTCATGCGGGATGCCGGGCTGGTGACGGTCTGCGAG
GAGGCGGCGTGCCCCAATATCGGGGAATGCTGGTCGCAGCGTCATGCCACGATGATGATCAT
GGGCGAGATCTGCACCCGGGCGTGTGCATTCTGCAATGTCACGACCGGACTGCCGAAGCATC
TCGATGAGGACGAGCCACGGCGGGTCGGAGAAGCGGTGGCGAAGCTTGGGCTCAAGCATGTC
GTCATCACTTCGGTGGACCGTGACGATCTGGAAGACGGGGGGGCGATGCACTTCGCGCGGGT
CATTCATGCGATCCGCGAGACCTCTCCACAGACCACGATCGAGATTCTGACGCCTGATTTCC
TGCGCAAGGATGGCGCGCTGGAGGTCGTGGTGGCGGCTCGGCCGGACGTTTTCAATCACAAT
ATCGAGACCATACCCCGGCTTTACCCCACCATCAGACCGGGTGCGCGTTACTATCAGTCCGT
CCGTCTCCTGGACGGAGTAAAGAAACTCGATCCTTCCATTTTTACCAAGTCCGGCCTGATGC
TTGGGCTTGGCGAGGAGCGGATGGAAGTGGCCCAGGTGATGGATGATTTCCGGATCGCGGAC
GTCGATTTCCTGACGCTTGGACAGTATCTCCAGCCGTCCGCGAAACATGCGGCGGTGGAAAA
GTTTGTTACCCCCGACGAGTTTGACGGCTATGCTGCTGCTGCCCGTTCAAAAGGATTTCTTC
AGGTCAGTGCCAGTCCGCTGACCCGGTCCTCCTACCATGCGGACAGTGATTTCGCGAAGCTT
CAGGCTGCGCGGAACAGCCGTCTGAAAGAAAGTCTCTGA
SEQ ID No. 2: LipB
Gene Accession ID: 3248567 (lip B) lipoyl transferase. (Source: NCBI)
Gluconobacter oxydans 621H, complete genome.
Coding nucleic acid sequence:
ATGCCGAAAACGAGGGCAATGACCCGAAATGAGATTTGTGAAGAAATTTTGTGGAAATCTTC
CCCGGGACTGACTCCCTACCCCGAGGCCCTGACCTTCATGGAGGAGCGTGCGCGGGCCATTC
ATCAGGGGACAGCAGAACCGCTGGTCTGGCTTGTCGAGCATCCTCCCGTCTTTACGGCAGGC
ACCTCCGCCAAAGATGCGGACCTCTACAATCCTCACGGCTACCCGACCTATTCCGCCGGACG
CGGCGGCCAGTGGACCTATCACGGACCGGGGCAAAGGCTCGGTTATGTCATGATGGATCTGA
CGAAGCCGAACGGCACCGTCCCGTCCCGCGACCTGCGCGCTTTTGTCGCGGGACTGGAAGGC
TGGATGACCGACACCCTCGCCCGGCTCGGCGTCACGGCCTTTACCCGAGAGGGCCGGATCGG
CGTCTGGACGATCGATCCCCTCACGGGCCTGGAAGCCAAGATCGGGGCGCTGGGCATCCGCG
TCAGCCGGTGGGTCAGCTGGCATGGCGTTTCGATCAATGTCAGTCCCGATCTGACAGATTTC
GATGGAATCGTGCCCTGCGGCATCCGCGAGTTCGGTGTCACCAGTCTCCAGCGATTCGACAG
CAGCCTGACGATGGCGGATCTCGATGACGCCCTCGCCGCCGCATGGCCCGGACGGTTCGGCT
CTATTCCGCAGGCGGCGTGA
SEQ ID No. 3: SufE
Gene Locus Tag GOX0268 (Source: NCBI)
Gluconobacter oxydans 621H, complete genome.
GeneID: 3248646 “SufE protein probably involved in Fe-S center assembly”
Gluconobacter oxydans 621H
GTGAGTGATGCCTATCTTGTTCCCCAGGAGGACACGGCTGCTGCGGCTATCGAGGAGATCGA
GGCCGAGCTGGGTCTGTTTGATGACTGGATGGAGCGGTATCAGTACATCATCGAGATGGGAC
GCAAGCTGCCGCCATTTCCGGAAGAGTGGCAGGATGATGCCCATCGGGTTCCGGGCTGTCAG
AGCCAGGTCTGGCTTGAAGCGGTCGAACGGGATGGAAAGCTGTTTTTCGCCGGGGCGTCGGA
TGCCGCGATCGTTCAGGGGCTCGTGGCGCTTCTGCTGAGGGTTTATTCCGGCCGTCCGAAAT
CGGAAATTCTGGGAACAAGCCCTGTGTTTCTGCATGACATGGGACTGGTCAAGGCGCTTTCG
ACGAACCGTGGCAACGGGGTCGAGGCTATGGCGCAGGCCATTCAGAAGCGCGCGTCACACTA
G
SEQ ID No. 4, codon-optimized sequence for lipA:
ATGTCCCAGCGCATCACGATCGACCATCGCTCCGCCCCGGCCCTGCGCCATCCGGAAAAGGC
CCATCGCCCGGACAACCCGATCCAGCGCAAGCCGTCCTGGATCCGCGTCAAGGCCCCGAACC
ATCCGGTCTATCATGAAACGCGCGCCGTCATGCGCGACGCCGGCGTCGTCACGGTCTGCGAA
GAAGCCGCCTGCCCGAACATCGGCGAATGCTGGTGCCAGGTCCATGCCACGATGATGATCAT
GGGCGAAATCTGCACGCGCGCCTGCGCCTTCTGCAACGTCACGACGGGCGTCCCGAAGCATG
TCGACGAAGACGAACCGCGCCGCGTCGGCGAAGCCGTCGCCAAGCTGGGCGTCAAGCATGTC
GTCATCACGTCCGTCGACCGCGACGACGTCGAAGACGGCGGCGCCATGCATTTCGCCGGCGT
CATCCATGCCATCCGCGAAACGTCCCCGCAGACGACGATCGAAATCGTCACGCCGGACTTCG
TCCGCAAGGACGGCGCCGTCGAAGTCGTCGTCGCCGCCGGCCCGGACGTCTTCAACCATAAC
ATCGAAACGATCCCGGGCCTGTATCCGACGATCCGCCCGGGCGCCCGCTATTATCAGTCCGT
CCGCGTCGTCGACGGCGTCAAGAAGGTCGACCCGTCCATCTTCACGAAGTCCGGCGTCATGC
TGGGCCTGGGCGAAGAACGCATGGAAGTCGCCCAGGTCATGGACGACTTCGGCATCACCGAC
GTCGACTTCGTCACGCTGGGCCAGTATCTCCAGCCGTCCGCCAAGCATGCCGCCGTCGAAAA
GTTCGTCACGCCGGACGAATTCGACGGCTATGCCGCTGCTGCCCGTTCAAAAGGATTTCTTC
AGGTCAGTGCCAGTCCGCTGACCCGGTCCTCCTACCATGCGGACAGTGATTTCGCGAAGCTT
CAGGCTGCGCGGAACAGCCGTCTGAAAGAAAGTCTCTGA
SEQ ID No. 5, codon-optimized sequence for lipB:
ATGCCGAAGACGCGCGCCATGACGCGCAACGAAATCTGCGAAGAAATCCTGTGGAAGTCCTC
CCCGGGCGTCACGCCGTATCCGGAAGCCGTCACGTTCATGGAAGAACGCGCCGGCGGCATCC
ATCAGGGCACGGCCGAACCGGGCGTCTGGCTGGTCGAACATCCGCCGGTCTTCACGGCCGGC
ACGTCCGCCAAGGACGCCGACGTCTATAACCCGCATGGCTATCCGACGTATTCC241GCCGG
CCGCGGCGGCCAGTGGACGTATCATGGCCCGGGCCAGCGCGTCGGCTATGTCATGATGGACG
GCACGAAGCCGAACGGCACGGTCCCGTCCCGCGACCTGCGCGCCTTCGTCGCCGGCGTCGAA
GGCTGGATGACGGACACGGTCGCCGGCGTCGGCGTCACGGCCTTCACGCGCGAAGGCGGCAT
CGGCGTCTGGACGATCGACCCGGTCACGGGCGGCGAAGCCAAGATCGGCGCCGGCGGCATCC
GCGTCGCCGGCTGGGTCGCCTGGCATGGCGTCTCCATCAACGTCTCCCCGGACGGCACGGAC
TTCGACGGCATCGTCCCGTGCGGCATCCGCGAATTCGGCGTCACGTCCGTCCAGCGCTTCGA
CGCCGCCGGCACGATGGCCGACGTCGACGACGCCGTCGCCGCCGCCTGGCCGGGCGGCTTCG
GCTCCATCCCGCAGGCCGCCTGA
SEQ ID No. 6, codon-optimized sequence for sufE:
GTCTCCGACGCCTATCTGGTCCCGCAGGAAGACACGGCCGCCGCCGCCATCGAAGAAATCGA
AGCCGAACTGGGCCTGTTCGACGACTGGATGGAACGCTATCAGTATATCATCGAAATGGGCC
GCAAGCTGCCGCCGTTCCCGGAAGAATGGCAGGACGACGCCCATCGCGTCCCGGGCTGCCAG
TCCCAGGTCTGGCTGGAAGCCGTCGAACGCGACGGCAAGCTGTTCTTCGCCGGCGCCTCCGA
CGCCGCCATCGTCCAGGGCCTGGTCGCCCTGCTGCTGCGCGTCTATTCCGGCCGCCCGAAGT
CCGAAATCCTGGGCACGTCCCCGGTCTTCCTGCATGACATGGGCCTGGTCAAGGCCCTGTCC
ACGAACCGCGGCAACGGCGTCGAAGCCATGGCCCAGGCCATCCAGAAGCGCGCCTCCCATTA
G
SEQ ID No. 7: GOX04504-476661 (Putative transmembrane oxidoreductase)
ATGACGACCGGAAACATCCCAGACCCGTCATCGCCTGCGAAAGGCCCGGTCTGCATCATCGG
CGCCAGCGGCCGCTCAGGATCCGCGCTCTGCCGCGCGCTTCTGGCTGAAGGCCAGAAGATCA
TTGCCGTCGTGCGCAGCCAGGGCAATCTCGCGCCCGACATCGCAGAAGCCTGTCAAGCCGTG
CGGATCGCAGACCTTACGGACAGCGCCACGCTGGCTCTGGCGTTCGAAGATGCGGCGGTTAT
CGTCAACACGGCCCATGCGCGACACTTGCCCGCCATTCTGGCCGCTACGAAAGCCCCTATCG
TAGCGCTGGGCAGCACACGCAAATTCACGCGCTGGCCCGACGATCACGGACGGGGTGTCCTC
GCAGGCGAAACCGCCCTCAAGGCCGACGGTCGCCCGTCGATCATCCTGCATCCGACCATGAT
CTATGGTGCCCAGGGCGAAGACAATGTGCAGCGGCTAGCGAAGCTGCTGGAGCGCCTGCCCG
TCATCCCACTTCCTGGCGGTGGCCGTGCCCTCGTGCAGCCCATCGACCAGCGGGACGTTACA
CGCTGCCTGGTCTCGGCCATACATCTGATCCAGAACGGAGACGTCACGGGGCCGGAAAGCAT
CGTGATCGCCGGTCCGACAGCGGTGGCTTACCGGACCTTCGTGCGCATGGTGCTGTATTTTG
CGGGACTTGGCGGCCGTCCCATCGTCTCCCTGCCGGGATGGATGCTCATGGCGCTGTCTCAT
CTGACGCGGCATATCCGCAGACTGCCGCAGATCGCGCCGGAAGAAATCCGCCGCCTTCTGGA
AGACAAGAATTTCGATGTGGGTCCGATGGAACAGCGTCTGGGCGTCACGCCCGTTCCGCTTG
CCAACGGGCTGCACCATCTGTTCGGCAACAGAACGCGCCAGAAGCAGGAGCCCTGA
SEQ ID No. 8: GOX0586-620902 (Membrane-bound aldehyde dehydrogenase,
small subunit)
ATGACCACGAAATTTGAACTCAACGGACAGCCCGTTACGGTCGACGCCCCGGCAGACACCCC
CCTGCTGTGGGTCATCCGTGACGACCTGAACCTGACCGGCACCAAGTTCGGCTGCGGCATCG
GCGAATGCGGTGCCTGCACCGTCCATGTGGGCGGCCGCGCCACGCGCTCCTGCATCACGCCG
CTCTCCGCCGTCGAAGGCGCTTCGATCACCACGATCGAAGGCCTCGACCCGGCAGGAAACCA
CGTCGTGCAGGTCGCCTGGCGTGACCAGCAGGTGCCGCAGTGCGGCTACTGCCAGTCCGGCC
AGATCATGCAGGCCGCAAGCCTCCTGAAGGACTATCCGAACCCGACGGACGACCAGATTGAC
GGCGTAATGGGCGGCAGCCTCTGCCGCTGCATGACCTATATCCGCATCCGCAAGGCCATCAA
GGAAGCTGCCTCAAGGCAGCAGGAAGGCGCCAACAATGGCTAA
SEQ ID No. 9: GOX0973-Outer membrane channel lipoprotein
TCAGAAGCGGTATTCGATACCTGCGCCAACGACGGTCGGGTCGAGGGAATCATGCGCCGTGA
TCTTCGTCGTCTCGGCACTGTCCTGGGCCCAGACGTGGACACGCATGAACATCTGCTTCACG
TCGAAGTTGAAGAACCAGTTGCCGACGACCTGGTAGTCGAAGCCTACATTGACGGACGGGCC
ACCGGTCACACCGATGTTCACCTTCTGCGTCAGGCCATTGTTGGCCGGAGAGATGTCGTGGA
ACCATGCCAGCGTTGCGCCAACACCCACATACGGATTGAAGCGCTTGTGCGGGCGGAAGTGC
CAGGCGAACGTGACCGTCGGCGGCAGAACCCAGGCGCTGCCAACATCGACCTTGCCCAGACC
CGGAACGCCTTCAGCGGCGATCTCGTGACGGGTGCTGGCGGCGATCAGGTCAACCGACAGGT
TATCCGTGAAGAAATATTCGAACGTCAGTTCCGGCATGACCTGACGCGTCGTCTTCACGCGG
CCACCGAGATGGGCGCCGTTCAGGGACAGGCTGCTGTCACGGTCTTCCGGCAGAACGCCGAG
AGCGGACAGACGGACGATGAAGTCGCCCTTGCCGAGTCCGATGCGCGTGTTGGCGCAGGTCT
GGAACAGGCCGCAGCGACCCGAAGCCGGCGCATGGATGTTCACGGAAGGCACCGGAGCCGCC
GTCATGGGAGCACTGACCATGACCTGAGGAGCAGCAGGAGCAGTTGCCGGGGCGGTCTGGGC
AGGGACCGTCTGGGCAAACGCCGGAGTTGCAACAACACCGACTGCGGCAGCCAGCGCAAAAG
CGTTAAATTTCAT
SEQ ID No. 10, LipA protein sequence:
MSQRITIDHRSAPALRHPEKAHRPDNPIQRKPSWIRVKAPNHPVYHETRALMRDAGLVTVCE
EAACPNIGECWSQRHATMMIMGEICTRACAFCNVTTGLPKHLDEDEPRRVGEAVAKLGLKHV
VITSVDRDDLEDGGAMHFARVIHAIRETSPQTTIEILTPDFLRKDGALEVVVAARPDVFNHN
IETIPRLYPTIRPGARYYQSVRLLDGVKKLDPSIFTKSGLMLGLGEERMEVAQVMDDFRIAD
VDFLTLGQYLQPSAKHAAVEKFVTPDEFDGYAAAARSKGFLQVSASPLTRSSYHADSDFAKL
QAARNSRLKESL
SEQ ID No. 11, LipB protein sequence:
MPKTRAMTRNEICEEILWKSSPGLTPYPEALTFMEERARAIHQGTAEPLVWLVEHPPVFTAG
TSAKDADLYNPHGYPTYSAGRGGQWTYHGPGQRLGYVMMDLTKPNGTVPSRDLRAFVAGLEG
WMTDTLARLGVTAFTREGRIGVWTIDPLTGLEAKIGALGIRVSRWVSWHGVSINVSPDLTDF
DGIVPCGIREFGVTSLQRFDSSLTMADLDDALAAAWPGRFGSIPQAA
SEQ ID No. 12, SufE protein sequence:
VSDAYLVPQEDTAAAAIEEIEAELGLFDDWMERYQYIIEMGRKLPPFPEEWQDDAHRVPGCQ
SQVWLEAVERDGKLFFAGASDAAIVQGLVALLLRVYSGRPKSEILGTSPVFLHDMGLVKALS
TNRGNGVEAMAQAIQKRASH

1. (canceled)

2. An acid-tolerant microorganism comprising a nucleic acid sequence that:
(i) hybridizes under stringent conditions to the nucleic acid of SEQ ID No. 4 and the nucleic acid encodes a protein able to convert a synthetic tetrapeptide substrate, containing an N(epsilon)-octanoyl lysine residue, corresponding in sequence to the lipoyl binding domain of the E2 subunit of pyruvate dehydrogenase at a rate at least 50% of that of wild-type LipA,
(ii) hybridizes under stringent conditions to the nucleic acid of SEQ ID No. 5, and the nucleic acid encodes a protein that transfers an octanoyl group from octanoyl-ACP to apo-H protein at a rate at least 50% of that of wild-type LipB, or
(iii) hybridizes under stringent conditions to the nucleic acid of SEQ ID No. 6 and the nucleic acid encodes a protein that binds SufB with a dissociation constant no more than twice the value of the dissociation constant of SufB and wild-type SufE.

3-4. (canceled)

5. The microorganism of claim 2, wherein the microorganism is a bacterium of the genus Gluconobacter.

6. The microorganism of claim 5, wherein the microorganism is Gluconobacter oxydans.

7. (canceled)

8. The microorganism of claim 2, wherein at least one of said nucleic acid sequences is in a vector.

9. The microorganism of claim 8, wherein the vector comprises at least one of an additional lipoic acid synthesis gene, a selectable marker, a transcription terminator, an origin of replication, and a promoter.

10. The microorganism of claim 9, wherein the additional lipoic acid synthesis gene is sufE.

11-12. (canceled)

13. The microorganism of claim 2, wherein the nucleic acid is present in multiple copies in the microorganism.

14-22. (canceled)

23. A vector for producing lipoic acid in a microorganism, comprising a nucleic acid sequence that:
(i) hybridizes under stringent conditions to the nucleic acid of SEQ ID No. 4, and the nucleic acid encodes a protein able to convert a synthetic tetrapeptide substrate, containing an N(epsilon)-octanoyl lysine residue, corresponding in sequence to the lipoyl binding domain of the E2 subunit of pyruvate dehydrogenase at a rate at least 50% of that of wild-type LipA,
(ii) hybridizes under stringent conditions to the nucleic acid of SEQ ID No. 5 and the nucleic acid encodes a protein that transfers an octanoyl group from octanoyl-ACP to apo-H protein at a rate at least 50% of that of wild-type LipB, or
(iii) hybridizes under stringent conditions to the nucleic acid of SEQ ID No. 6 and the nucleic acid encodes a protein that binds SufB with a dissociation constant no more than twice the value of the dissociation constant of SufB and wild-type SufE.

24-25. (canceled)

26. The vector of claim 23, further comprising an additional lipoic acid synthesis gene.

27. The vector of claim 23, wherein the additional lipoic acid synthesis gene is an Fe—S cluster assembly gene.

28. The vector of claim 27, wherein the Fe—S cluster assembly gene is sufE.

29-33. (canceled)

34. A method of producing lipoic acid, comprising culturing in a culture medium an acid-tolerant microorganism comprising a nucleic acid sequence that:
(i) hybridizes under stringent conditions to the nucleic acid of SEQ ID No. 4, and the nucleic acid encodes a protein able to convert a synthetic tetrapeptide substrate, containing an N(epsilon)-octanoyl lysine residue, corresponding in sequence to the lipoyl binding domain of the E2 subunit of pyruvate dehydrogenase at a rate at least 50% of that of wild-type LipA,
(ii) hybridizes under stringent conditions to the nucleic acid of SEQ ID No. 5 and the nucleic acid encodes a protein that transfers an octanoyl group from octanoyl-ACP to apo-H protein at a rate at least 50% of that of wild-type LipB, or
(iii) hybridizes under stringent conditions to the nucleic acid of SEQ ID No. 6 and the nucleic acid encodes a protein that binds SufB with a dissociation constant no more than twice the value of the dissociation constant of SufB and wild-type SufE.

35-36. (canceled)

37. The method of claim 34, wherein the microorganism is a bacterium of the genus Gluconobacter.

38. The method of claim 37, wherein the microorganism is Gluconobacter oxydans.

39-46. (canceled)

47. The method of claim 34, wherein the medium further comprises an agent that induces gene expression.

48. The method of claim 47, wherein the agent is selected from the group consisting of octanoic acid, tetracycline, galactose, IAA, IPTG, arabinose, and nalidixic acid.

49. The method of claim 34, wherein the medium further comprises a precursor of lipoic acid.

50. The method of claim 49, wherein the precursor is octanoic acid, octanoate, octanoic esters, caprylic aldehyde, alcohol, a carbohydrate, or an octanoylated molucule such as octanoyl-AMP.

51-55. (canceled)

56. The method of claim 34, wherein the lipoic acid is isolated from the culture medium.

57-60. (canceled)

61. The method of claim 34, wherein the lipoic acid isolated is R-lipoic acid and is essentially free of S-lipoic acid.

62-67. (canceled)
